(19) United States
(12) Patent Application Publication
(10) Pub. No.: US 2023/0245726 A1
(43) Pub. Date: Aug. 3, 2023

(54) METHOD FOR ASCERTAINING A PRODUCT COMPOSITION FOR A MIXED CHEMICAL PRODUCT

(71) Applicant: COVESTRO INTELLECTUAL PROPERTY GMBH & CO. KG, Leverkusen (DE)

(72) Inventor: Thomas FÄCKE, Leverkusen (DE)

(21) Appl. No.: 17/049,607

(22) PCT Filed: Apr. 17, 2019
(86) PCT No.: PCT/EP2019/060001

§ 371 Date: Oct. 22, 2020

(30) Foreign Application Priority Data
Apr. 23, 2018 (EP) 18168734.4

(51) Int. Cl. G16C 20/30; C08G 18/75; C08G 18/42; C08G 18/62; C08G 18/40; C08G 18/32; C08G 18/66; C08G 18/65; G16C 20/70

(52) U.S. Cl. CPC G16C 20/30; C08G 18/755; C08G 18/4277; C08G 18/6208; C08G 18/4063; C08G 18/3206; C08G 18/664; C08G 18/6511; G16C 20/70

(57) ABSTRACT

The invention relates to a method for ascertaining a product composition for a mixed chemical product, a series of feature values, which numerically describe feature values in each case of a descriptor of the particular mixed product, being provided, for each first product composition of a plurality of first product compositions, in the case of a plurality of first product compositions for a particular mixed chemical product, each first product composition being characterised by a numerical product distribution for describing the proportions of components of the first product composition, the series of feature values for each mixed product being mapped by first bijective mapping onto a series of mapped feature values, a series of test feature values, which numerically describe in each case a behaviour property of the particular mixed product, being provided for a plurality of second product compositions for a particular mixed chemical product, the series of test feature values for each mixed product being mapped by second bijective mapping onto a series of mapped test feature values, at least the first or the second mapping including a modification, each series of mapped test feature values of a second product composition being assigned to a series of modified feature values of a first product composition, a correlation matrix being ascertained by multivariate analysis of the associated series, a target requirement profile being predefined for describing at last one behaviour property of a target mixed product and a target descriptor profile for describing descriptors of a target product composition being determined on the basis of the target requirement profile and the correlation matrix.

The invention relates to a method of ascertaining a product composition for a mixed chemical product, to a method of producing a mixed chemical product from a product composition, and to a mixed chemical product.

Plastics are used nowadays in virtually countless applications. Since the commencement of their development in the mid-20th century, various material classes and technologies have become established. A distinction is made here between thermoplastic bulk plastics, for example polyethylene (PE), polypropylene (PP), polyester (PET, i.e. polyethylene glycol terephthalate, PBT, polybutylene terephthalate), polyvinylchloride (PVC), and thermosets, for example epoxy resins, phenolic resins, vulcanizable rubber mixtures, unsaturated polyester/styrene mixtures, silicones or polyurethanes. The individual material classes have each been able to develop their own markets and applications, which is usually understandable on account of their adequate material properties and price points.

The economic aspect limits the selection of these materials, even though there is an immediate great need for new material properties. The aim here is usually not just to fulfill a specific property requirement; instead, there is a desire to simultaneously fulfill a multitude of, often more than three, properties simultaneously.

The abovementioned thermoplastics are usually unsuitable for such demands since product adjustment generally does not fit with the scale effects of these bulk plastics. For that reason, material technologies that have inherent availability of mutually combinable components and simultaneously meet cost demands are selected for such cases.

System chemistries in widespread use are the phenolic resins, vulcanizable rubber mixtures, silicones, epoxy systems and polyurethanes, all of which are also available as reactive systems and in that sequence enable increasingly greater flexibility of the establishment of the profiles of properties. Polyurethanes in particular show enormous adaptation capacity. This is associated with the fact that there are numerous isocyanates and isocyanate-reactive components in this technology. Moreover, these also exist as one-component PU materials, and so reference is made here to mixed chemical products. This variety constitutes a particular opportunity but also a challenge to the developer, since it is difficult to find the best selection of components, especially since process conditions are often also of significance. In industrial practice, an iterative process based on trial and error has become established, generally commencing with a stepwise improvement proceeding from an obvious reference composition. However, this methodology has multiple problems since local optima are found here, and further alternatives are fundamentally not taken into account.

A further problem here is that, even if the requisite information to ascertain a suitable product composition is basically available, this is frequently in the possession of different parties and constitutes trade secrets worthy of protection. For instance, the supplier of a product composition as starting material knows its composition. The customer, by contrast, has used the product composition for production of a mixed product and knows characteristic values of the mixed product. For the party in question or the company in question, there is a considerable risk in passing this corresponding information on in unfiltered form to the respective other party, since, for example, the customer can then request the known product composition from an alternative supplier. Conversely, the supplier can also draw sensible conclusions from the test values and take account of the corresponding results in the supplying of competitors to the customer. In such a situation, the sharing of the corresponding information would basically lead to the ascertaining of the correct product composition, which would obviously be commercially advantageous to both parties, but the above aspects constitute important hindrances or even exclusion criteria.

It is therefore an object of the present invention to be able to ascertain a product composition being sought for a mixed chemical product without disadvantageous distribution of sensitive data, and to be able to produce such a mixed chemical product.

This object is achieved by a method of ascertaining a product composition for a mixed chemical product having the features of claim 1, by a method of producing a mixed chemical product from a product composition having the features of claim 14, and by a mixed chemical product having the features of claim 15.

The invention is based on the finding that both data of the supplier's product compositions and test data of the customer's mixed chemical products can be released in a comparatively problem-free manner if not just the assignment of the numerical values to corresponding parameters is removed, but the numerical values themselves are also subjected to bijective mapping or function.

This is because, even if the explicit assignment to particular parameters is not communicated, it is regularly apparent to the person skilled in the art from the numerical values themselves what kind of parameter must be involved. But if the numerical values themselves are altered by a bijective function, this manner of discernibility is lost, but correlations are maintained.

The method proposed serves to ascertain a product composition for a mixed chemical product, wherein a multitude of feature values, each of which numerically describes a descriptor of the particular mixed product, is provided for each of a multitude of first product compositions for a particular mixed chemical product, wherein each first product composition is characterized by a numerical product distribution for description of proportions of components of the first product composition. The product composition is for a mixed chemical product in the sense that the product composition is a starting material for the mixed chemical product. It is preferable that each series of feature values describes the same series of descriptors. Two or more feature values that describe the same descriptor may be described as being of identical type. A descriptor is understood to mean a calculable, measurable or otherwise determinable property of a product composition as substance. Such a property is preferably independent of any further processing of the product composition. What is meant here by numerical description is that the corresponding parameter is described quantitatively—i.e. by a number.

In the method proposed, the series of feature values for each mixed product is mapped by a first bijective mapping onto a series of mapped feature values.

Further, in the method proposed, a series of test feature values, each of which numerically describes a behavior property of the particular mixed product, is provided for a multitude of second product compositions for a particular mixed chemical product. It is preferable that each series of test feature values describes the same series of behavior properties. A behavior property is understood to mean a calculable, measurable or otherwise determinable property of a product composition as substance. Such a behavior property may also include processing properties of the product composition.

In the method proposed, the series of test feature values for each mixed product is mapped by a second bijective mapping onto a series of mapped test feature values.

According to the proposal, at least the first or second mapping includes a variation. This means that at least the first or second mapping includes a variation in the values mapped—i.e. in the feature values or the test feature values. At least one of the first or second mappings is therefore not an identity mapping.

In the method proposed, each series of mapped feature values of a first product composition is assigned to a series of mapped test feature values of a second product composition. This can also be expressed in that this second product composition is assigned to that first product composition, or vice versa—which means the same thing here.

In the method proposed, a multivariate analysis of the assigned series determines a correlation matrix between the mapped feature values and the mapped test feature values. The correlation matrix may also include gaps, but is preferably complete. The correlation matrix preferably comprises elements, preferably including numbers, and these elements qualitatively or—as is preferred—quantitatively describe a particular correlation between the mapped test feature values and the mapped feature values. The multivariate analysis of the assigned series corresponds to a multivariate analysis of the mapped feature values and the mapped test feature values.

In the method proposed, a target profile of requirements for description of at least one behavior property, preferably of multiple behavior properties, of a target mixed product is defined and, on the basis of the target profile of requirements and the correlation matrix, a target descriptor profile for description of descriptors of a target product composition is determined.

If a target descriptor profile, for example with specific feature values for the descriptors of a target product composition, is known with greater or lesser accuracy, the product distribution for this target product composition can be determined in various ways, for example by recourse to the product distributions of product compositions having known feature values for description of these descriptors.

It is therefore preferable that the determining of the target descriptor profile comprises, based on a comparison of the target descriptor profile with the feature values of the first product compositions of the multitude, determining a first product composition of the multitude as starting product composition and varying the product distribution of the starting product composition on the basis of the feature values of the remaining first product compositions of the multitude to obtain the target product composition.

Further options and further developments are described below.

The term “bijective mapping” may also be regarded as a bijective function in the mathematical sense. Bijective means that the mapping or function is both surjective and injective.

The description of a product composition of a mixed product by a numerical distribution—here: product distribution—of proportions of the components of the product composition follows the ingredients of a formulation that are customary in the corresponding industry:

For paints, these are typically one or more components selected from binders, optionally one or more crosslinkers, primer-surfacers, pigments and dyes, leveling aids, defoamers, degassers, adhesion improvers, stabilizers such as antioxidants and light stabilizers, dispersing and/or rheology additives, surface modifiers, solvents and/or water, cosolvents, (dye) masterbatches, initiators and/or catalysts, anticorrosion additives.

For adhesives and sealing compounds, these are binders (e.g. semicrystalline polymers for hotmelt adhesives, dispersions for aqueous systems), solvents, tackifiers, crosslinkers, catalysts, plasticizers, stabilizers, thixotropic agents, accelerators, and dyes or fillers.

For casting resins, these are binders and crosslinkers, flame retardants, catalysts, plasticizers, stabilizers, accelerators, and dyes or fillers and additives.

For foams, these are polyol and isocyanate, blowing agents, water or air, catalysts, stabilizers, accelerators, fillers, flame retardants and additives.

The description of the product composition of the mixed product may also relate to, or indirectly describe, the chemical makeup of an individual component. This procedure is an option especially when the individual component is to be optimized. Individual components mean the components of a paint, an adhesive, a casting resin or foam.

Reference may be made here to the base components (monomer compositions) and/or the chemical functional groups (e.g., alcohol, acid, ether, ester, carbonate, urethane, urea, methylene group, aromatic or aliphatic ring structures), molecular weight distribution, solids contents, functionality or superstructures such as hard segments, hydrogen bond formers, main or side chain assignment, etc.

The product distribution of a product composition is the quantitative composition of the individual components of the product composition. Particular ranges of amounts are typically chosen here: The primer-surfacers, anticorrosion additives and pigments of a formulation determine the total proportions and are at a total proportion of 1-60%. Additives such as leveling aids, defoamers, degassers, adhesion improvers, stabilizers such as antioxidants and light stabilizers, dispersing and/or rheology additives, surface modifiers, initiators and/or catalysts are usually used at less than 5%, preferably less than 1%. Solvents and water are used at up to 60% in order to adjust viscosities. Binders and crosslinkers, according to their ratio of equivalents to one another, have a fixed ratio and may account for a total of up to 50% (in the case of filled systems) and up to 99% in the case of unfilled systems.

In the chemical description of the components, the product description may be more detailed and in that case relates to the weight ratios, equivalents ratios or molar ratios of the base constituents, functional groups and/or superstructures.

The product distribution may either be known from laboratory methods or likewise also be determined by experimental methods. Suitable methods are especially those capable of determining absolute proportions, for example NMR spectroscopy, or else methods that can determine relative ratios. In general, methods such as UV, IR or Raman spectroscopy, mass spectrometry, gas, liquid or gel permeation chromatography methods, but also titration methods, are suitable. It is also possible to dispense entirely with an absolute concentration or one determined by calibration. This is the case especially for spectroscopic/spectrometric methods and chromatographic methods. Calibration can be effected here with the features of a chromatogram and/or spectrogram/spectrum, for which it is additionally possible to use digital training methods (neural networks, decision trees or the like).

It is possible that a first product composition is not identical to its assigned second product composition. Instead, one product composition may preferably be derived from the respective other product composition. It may thus be the case that the difference between two assigned product compositions consists of a particular addition of a substance. This added substance may consist of a multitude of substances in a particular composition. The addition may be present in the first product composition with respect to the second product composition, or vice versa. Preferably, a constant and identical addition exists as a difference between all the respectively assigned product compositions. Preferably, this addition is less than 70 percent by weight, more preferably less than 50 percent by weight and most preferably less than 20 percent by weight, based on the overall composition.

A further preferred embodiment of the method is characterized in that the series of varied feature values of a second polymer composition is assigned to that series of varied feature values of a first product composition in which the second product composition is essentially identical to the first product composition. In other words, a second product composition is assigned to that first product composition to which it is essentially identical.

In a preferred embodiment of the method, the series of feature values for each first product composition is provided on a first computer system on which the first bijective mapping is executed, in that the series of test feature values for each second product composition is provided on a second computer system on which the second bijective mapping is executed, and in that the first computer system and the second computer system are encompassed by a respectively disjoint intranet. This means that the provision and mapping of the data is effected in mutually separate computer systems. In other words, there is a first intranet comprising the first computer system, and a second intranet comprising the second computer system, with no intersection between the first intranet and the second intranet. Consequently, for the data connection between the first intranet and the second intranet, it is necessary to leave both intranets, such that such a connection has to be made via an outside network with respect to the two intranets, for example the internet.

In a further preferred embodiment of the method, the determination of the target descriptor profile is performed at least partly on the first computer system.

A preferred embodiment of the method is characterized in that the multivariate analysis is executed on a third computer system encompassed by an intranet that is disjoint from the respective intranet of the first computer system and the second computer system. In this variant, an effectively “neutral” third computer system is thus provided, which is separate both from the first computer system and from the second computer system. It is also possible that the multivariate analysis is performed on the first computer system and separately on the second computer system, and then is the subject of comparative discussion.

A further preferred embodiment of the method is characterized in that the product distribution and the series of feature values for each first product composition are stored by data encapsulation in the first computer system with respect to the second computer system, and in that the series of test feature values for each second product composition is stored with data encapsulation in the second computer system with respect to the first computer system. Such a data encapsulation means that technical access barriers are implemented in the system in question with respect to the system to be encapsulated, which permit active data access only by specific authorization. Such technical access barriers may also exist with respect to all accesses from outside the intranet in question. Thus, there is not just separation of the data in different computer systems, but also active shielding of the data from one another.

In a preferred embodiment of the method, the series of mapped feature values for each first product composition is transmitted from the first computer system to a target computer system of a disjoint intranet. In principle, this may be any target computer system. It is preferable that it is transmitted to the second computer system or the third computer system.

With regard to the ascertaining of a product distribution for the target product composition, in a further preferred embodiment of the method, in a calculation model provided, input of feature values for description of a particular descriptor results in output of a product distribution of a product composition for a mixed product for approximation of the feature values, and the target descriptor profile is input into the calculation model for output of the target product composition. In other words, the calculation model offers the possibility of determining a product distribution from desired feature values, the product composition of which has the desired feature values. It then follows from the calculated correlation with the behavior properties that the mixed chemical product also has the desired test feature values. Preferably, the calculation model is provided and, alternatively or additionally, the target descriptor profile is input in the first computer system.

In principle, the calculation model may have been determined in any desired manner. A preferred embodiment of the method that builds thereon is characterized in that the calculation model is ascertained at least partly by multivariate analysis, preferably executed in the first computer system, of the series of feature values of each first product composition with respect to the product distribution of this product composition. As well as this multivariate analysis, it is also possible for analytical considerations and formulae to be included in the ascertaining of the calculation model.

In principle, the feature values for description of a particular descriptor may be determined in any desired manner. A further preferred embodiment of the method is characterized in that, for each first product composition of the multitude, the series of feature values is ascertained at least partly, preferably completely, by a calculation based on the corresponding product distribution. Thus, the feature values are not based purely on measurements. It is further preferable that the calculation is based on a physical calculation model based on the product distribution. This calculation model may also form a partial or complete basis for ascertaining the above calculation model.

The feature values for description of the particular descriptors can be ascertained via various methods, preference being given to stoichiometric methods, topological and structural methods, physicochemical methods, quantum-chemical methods or specific indices.

Stoichiometric methods are understood to mean methods that calculate the composition of base constituents, functional groups, solids contents or superstructures on the basis of weight/equivalent/molar ratios. This generates feature values for descriptors, for example urethane density (in grams per kilogram or mol per kilogram of the product composition) or the proportion of a monomer, for example the amount of any isocyanate used (in grams per kilogram or mol per kilogram of the product composition). In this case, the proportions in all components are determined and then based on the total amount.

Topological and structural methods are considered, for example, to be molecular weight distribution (mass or number average), network node density, average network arc length or distribution thereof, the proportion of ring structures or other uncrosslinked components (sol), the proportion by weight of hard segment (as a portion of diisocyanate and diol of urethane-rich substructures that form), the corresponding phase components in the case of phase-separated materials, etc. For this purpose, it is possible to use experimental results such as molecular weight distribution by GPC, theoretical calculations such as Monte Carlo simulations, x-ray structure methods for determination of crystalline components.

Physicochemical methods are understood to mean methods of determining solubility characteristics, such as octanol-water coefficients, Hansen solubility parameters, dipole moment determination, surface charge properties (zeta potential), viscosity, surface tension (tensiometry), which are generally ascertained or performed experimentally. It is possible to relate the physicochemical methods to the individual components. It is likewise possible to relate these to structural subgroups of the components. In that case, a combination of stoichiometric, topological and physicochemical methods is utilizable, in which case the physicochemical experimental methods can be applied to model compounds and then approximated to the structural subgroups.

Quantum-chemical methods are understood to mean methods of calculating partial charges, polarizability, dipole moments, orbital energies etc., which are calculated by means of quantum-chemical calculations by ab initio methods, semiempirical methods, density functional theory.

Specific indices are especially those that are derived from a chemical composition or preparation and enable the person skilled in the art to make a direct link with his actions. Typical examples in the case of polyurethanes are the equivalents ratio of isocyanate to alcohol, the degree of chain extension and neutralization level in the case of polyurethane dispersions or soft segment content, and in the case of epoxy systems the equivalents ratio between epoxy amine or epoxy acid, epoxy oligomer distribution, epoxy-binder ratio etc.

It may be the case in principle that the first bijective mapping maps each input value on its own and independently of the other input values. In other words, every altered feature value is then dependent solely on a specific feature value prior to the variation. Alternatively or additionally, this may also be applicable to the second bijective mapping based on the feature values. In a preferred embodiment of the method, however, the first bijective mapping comprises a coordinate transformation of the series of feature values to the series of varied feature values. This is therefore a first bijective mapping that maps a series of input values—the feature values—onto an equally large number of starting values, in which case any starting value in principle may be dependent on one, more than one or all input values and bijectivity remains assured overall. Such a case is a coordinate transformation. It is further preferable that, alternatively or additionally and especially analogously, the second bijective mapping comprises a coordinate transformation from the series of test feature values to the series of varied test feature values.

In a further preferred embodiment of the method, the first bijective mapping and/or the second bijective mapping is a constant and strictly monotonous function with a continuously varying derivative.

Mapping by the bijective mappings for all product compositions follows the assembly thereof, with the arrangement of the product compositions remaining the same in each case and hence being comparable between the various feature values and the varied test feature values.

Examples of bijective, constant, strictly monotonous and selectively normalizing functions are described below, where the values may be the feature values or the test feature values.
1. In the case of solely positive values, the determination of the reciprocal of the greatest value of all product compositions in question and then the respective multiplication thereof by each individual value or alternatively additionally multiplication by 100 (in that case as a percent value).
2. The square of the respective value and subsequent multiplication by the reciprocal of the square of the greatest value of the product compositions in question.
3. The (decadic or natural) logarithm of the value.
4. The square root of the value.
5. The reciprocal of the value.
6. The determination of the arithmetic average and of the variation (sum of the squares of the differences of individual value minus arithmetic average—divided by the number of values considered). The bijective, constant, strictly monotonous function is then the difference of the value minus the arithmetic average—divided by the variance (student distribution).

A monotonous function means both monotonously rising and monotonously falling functions. It is also possible to transform individual values in a monotonously rising manner and other values in a monotonously falling manner.

Further bijective, constant, strictly monotonous and selectively normalizing functions are of course also conceivable. These may be applied to all values—i.e. in each case feature values or test feature values—or else solely to individual values. It is also possible in each case to apply a different bijective, constant, strictly monotonous and selectively normalizing function for each series of describing features or performance criteria.

The first bijective mapping may consist of a series of respectively descriptor-based sub-mappings that are each independent of one another. It is preferable here that the first bijective mapping comprises a one-dimensional bijective sub-mapping for each individual descriptor. It may be the case here that an identical bijective sub-mapping is effected for each feature value of the same type.

Correspondingly, it is preferable that the one-dimensional bijective sub-mapping of each descriptor is the same for every first product composition.

Alternatively, it is also possible to perform a different bijective sub-mapping for each of the feature values of the same descriptor of different first product compositions. It is also possible to use the same bijective sub-mapping for groups of feature values.

This is also analogously applicable to the second bijective mapping. Thus, it is preferable that the second bijective mapping comprises a one-dimensional bijective sub-mapping for each individual behavior property. It is further preferable that the one-dimensional bijective sub-mapping of each behavior property is the same for every second product composition. It may alternatively be different or the same only in groups.

According to the mathematical correlations of the feature values or the corresponding descriptors with respect to one another, of the test feature values or the behavior properties with respect to one another and of the feature values or the descriptors for the test feature values or the behavior properties with respect to one another, specific bijective, constant, strictly monotonous functions are of particularly good suitability, which leads to different high (i.e. close to 1.00) or low (i.e. close to −1.00) correlation coefficients. It is therefore advantageous to test multiple different bijective, constant, strictly monotonous mappings or functions in order to obtain maximum correlation coefficients which better describe dependence of the varied feature values on the varied test feature values with respect to one another and relative to one another. It is preferable then to use these correlation coefficients for the correlation matrix in question.

A preferred embodiment of the method is characterized in that the first bijective mapping includes a respective normalization function for the particular feature value and/or in that the second bijective mapping includes a respective normalization function for the particular test feature value. It may be the case that the starting endpoints of the respective normalization function of the first bijective mapping are determined by the range of the respective feature value for all first product compositions. It may likewise be the case that the starting endpoints of the respective normalization function of the second bijective mapping are determined by the range of the respective test feature value for all second product compositions.

The respective normalization function, as shown for the bijective, constant, strictly monotonous functions, can be effected by the determination of the reciprocal of the maximum value for the particular descriptor or the particular behavior property for all product compositions and multiplication thereof by each individual value to be mapped for the respective descriptor or the respective behavior property.

In addition, the optional normalization can also be effected by determining the maximum value Wₘₐₓ and the minimum value Wₘᵢₙ of all values Wᵢ of a descriptor or a behavior property of the product compositions. This minimum value is subtracted here from each value and then divided by the difference between maximum and minimum values—which in the present context is also referred to as range—see formula (1):

W_{i,norm}=(Wᵢ−Wₘᵢₙ)/(Wₘₐₓ−Wₘᵢₙ) (1)

As a result of the first mapping, the feature values mapped no longer directly describe the descriptors, but basically describe possibly merely fictional parameters that have arisen as a result of the application of the first mapping to the descriptors and are referred to here as “mapped descriptors” (Mᵢ). For analogous reasons, the mapped test feature values describe the “mapped behavior properties” (Pᵢ), with the two parameters referenced hereinafter merely by the abbreviation for the sake of simplicity.

The above multivariate analysis of the assigned series comprises at least a correlation calculation that also permits determination in the analyzable multidimensional space (with the variables of the mapped descriptors Mᵢ and the mapped behavior properties Pᵢ) of the dependences of the behavior properties on the descriptors through the calculation of the correlation coefficient r(Mⱼ,Pⱼ) between Mⱼ and Pⱼ. This is calculated by formula (2)

$$r ( M , P ) = \frac{{\sum }_{i = 1}^{n} ( {M}_{i} - \overline{M} ) ( {P}_{i} - \overline{P} )}{\sqrt{{\sum }_{i = 1}^{n} {( {M}_{i} - \overline{M} )}^{2}} \sqrt{{\sum }_{i = 1}^{n} {( {P}_{i} - \overline{P} )}^{2}}} \qquad ( 2 )$$

with n the number of product compositions, Mᵢ the mapped descriptors M, M the arithmetic average of M, Pᵢ the mapped behavior properties, P the arithmetic average of P.

As part of the ascertaining of the correlation matrix, it is possible to take account of the individual correlation coefficients, and classify them into three categories:
1. Mⱼ correlates positively with Pⱼ when r(Mⱼ,Pⱼ)>c,
2. Mⱼ correlates negatively with Pⱼ when r(Mⱼ,Pⱼ)<−c
3. Mⱼ does not correlate with Pⱼ when −c<r(Mⱼ,Pⱼ)<c

where c is the critical value (see also A. L. Socklogg, J. N Edney, Some extension of Student's t and Pearson's r central distributions, Technical Report (May 1972), Measurement and Research Center, Temple University, Philadelphia). For a confidence level of 0.05 (i.e. 95%), for example in the case of n=11, the result is a c=0.602.

Based on a target profile of requirements that describes the desired behavior properties of the target mixed product desired overall, the behavior properties are now considered individually in terms of their desired size. If large numerical values are desired in the application, the aim is to make the mapped feature values that correlate positively therewith likewise large, or the negatively correlating values are then made small and the non-correlating values may be chosen freely. If small numerical values are desired in the application, the aim is to make the mapped feature values that correlate positively therewith small, or the negatively correlated values are then made large and the non-correlating values in turn may be chosen freely.

It should be pointed out that a behavior property may depend on one or more descriptors and these possibly affect other behavior properties. It is also possible for two behavior properties to be influenced by one and the same descriptor. The aim here is thus to identify the matrix of the dependences from the correlation matrix and incorporate it into the planning.

Various situations and their solution are to be elucidated hereinafter.
P₁ and P₂ are both to be at a maximum (or simply larger) and M₁ correlates positively with both. In this case, the new composition(s) chosen for testing should be those in which M₁ likewise becomes greater.
P₁ and P₂ are both to be at a minimum (or simply smaller) and M₁ correlates positively with both. In this case, the new composition(s) chosen for testing should be those in which M₁ likewise becomes smaller.
P₁ and P₂ are both to be at a maximum (or simply larger) and M₁ correlates negatively with both. In this case, the new composition(s) chosen for testing should be those in which M₁ likewise becomes smaller.
P₁ and P₂ are both to be at a minimum (or simply smaller) and M₁ correlates negatively with both. In this case, the new composition(s) chosen for testing should be those in which M₁ likewise becomes greater.
One of the two of P₁ and P₂ is to be at a maximum (or simply larger) and the other at a minimum (or simply smaller) and M₁ correlates positively or negatively with both. When P₁ and P₂ do not have any further positive or negative correlations, one option is to develop new descriptors that are related to M₁ from a scientific point of view but appear to be sufficiently different. The aim is to choose the new descriptors M_{1-A}, M_{1-B}, . . . (i.e. derived properties of M₁) in such a way that at least one of these only correlates positively or negatively with P₁ or P₂.
P₁ is to be at a maximum (or simply larger) and P₂ at a minimum (or simply smaller) and M₁ correlates positively with both. If P₁ has a further positive correlation M₂, one option is to choose the new composition(s) to be tested in such a way that M₂ likewise becomes greater, but M₁ become smaller.

As becomes clear from the examples, the correlation matrix helps to identify the dependences and hence obtain an instruction for action by which behavior properties can be improved. If cross-dependences exist, it is possible to seek a balance (as shown in the last bullet). If the dependences are such that the target profile of requirements cannot be attained, the descriptors should be developed in a differentiated manner, in the hope of obtaining a correlation matrix that then correspondingly allows a new action.

In addition, it is also possible to consider the correlation coefficients between the descriptors or the Mᵢ or between the behavior properties or the Pᵢ themselves. If high significant values (i.e. greater than the critical value or smaller than the negative of the critical value) are found therein, it is also possible to utilize this to reduce the dimensions. It is thus seen very rapidly which descriptors affect one another and hence can also help to understand them more fully. One example is the two descriptors “urethane density” and “isocyanate monomer content”, which—according to the design of the study—frequently correlate directly with one another since urethanes are formed from an isocyanate.

Alternative methods of ascertaining dependence are likewise possible; particular mention should be made here of principal component analysis (PCA) (in this regard see also G. H. Dunteman: Principal Component Analysis. Sage Publications, 1989). This is a mathematical method that performs a coordinate transformation=main axis transformation with orthogonal axes, applying the criterion here that the first main component (mathematically formulated as linear combination of the individual features) has the greatest variance and the second main component has the second-greatest variance. The analysis involves checking which descriptors or Mᵢ or which behavior properties or Pᵢ themselves have high dependences and hence the correlation matrix can be reduced, or which descriptors or Mᵢ and behavior properties or Pᵢ form pairs of which one member can be removed for analysis in the correlation matrix in the multivariate analysis.

It is also possible that different bijective functions are examined, in which case different high (for positive correlation) and low (for negative correlation) values are obtained for a given pair of descriptor or Mᵢ and behavior property or Pᵢ. It is preferable then to use the maximum positive or minimum negative values for assessment in the multivariate analysis.

For calculation of the composition of the mixed target product—i.e. the target product composition—the variability of the test feature values with changes in product composition is analyzed. Preference is given here to conducting a classification of the compositions available using the same variation schemes. These may be, for example, the systematic alterations in the proportions of individual components, the alteration of chemical equivalents or other incremental alterations in product composition. These series with the same variation schemes then result in a consideration of the test feature values. Subsequently, for any available composition in the case of such a systematic alteration to any available composition, it is possible to predict the change in the property within a limited scope. For the calculation of the target product composition of the mixed target product, the correlating or determining descriptor identified in the multivariate analysis is then selected and its feature value that then corresponds to the test feature value of the mixed target product is determined. This can be effected in a simple manner, for example, with a graph representation in which the test feature values are plotted against the determining descriptor and then plotted with the same slope proceeding from the new reference position (i.e. one of the comparable compositions) with the variability of the test feature values (i.e. the slope in the curve). On attainment of the test feature value for the behavior property according to the target profile of requirements, it is possible to read off the value of the descriptor, by means of which the target descriptor profile can be built up. This value is then used to set the composition of the mixed target product in the computer with variation of the variation scheme in question, and to determine the target product composition.

Initially in the case of a multitude of product compositions that have been varied without any systematic variation scheme, it is possible to dispense with classification by variation scheme and utilize all datasets of product compositions. In this case, it is often advantageous to describe the variability of the test features as a function of more than one determining descriptor.

A preferred embodiment of the method is characterized in that the mixed chemical product comprises or consists of a phenolic resin, a vulcanizable rubber mixture, a silicone, an epoxy system and/or a solid foam, especially a polyurethane.

For the different mixed chemical products, epoxy resins, phenolic resins, vulcanizable rubber mixtures, unsaturated polyester/styrene mixtures, silicones or polyurethanes, descriptors for description by feature values that are particularly suitable in product developments with such material technologies are to be specified hereinafter.

The following descriptors are generally employable as content (as equivalent weight or weight percentage figure) for the mixed chemical products: carbon content, hydrogen content, oxygen content, nitrogen content, silicon content, sulfur content, halogen content, node point density (as equivalent weight).

Descriptors particularly suitable for vulcanizable rubber mixtures are (as equivalent weight or weight percentage figure) methylene groups, methyl side groups, unactivated double bonds, activated double bonds, styrene, sulfur content, crosslinking points, mercaptobenzothiazole content, dimethyldithiocarbamic acid content, tetramethylthiuram disulfide content, and vulcanization time and temperature.

Descriptors particularly suitable for silicones (as equivalent weight or weight percentage figure) are dimethylsilanoxy group content, diphenylsilanoxy group content, methylphenylsilanoxy group content, monomethylsilanoxy group content, monophenylsilanoxy group content, trimethylsilanoxy group content, silicone acrylate group content, titanium tetrabutoxide content, silane group content, allylsilyl group content, aminopropylsilyl group content.

Descriptors particularly suitable for epoxides and phenolic resins are (as equivalent weight or weight percentage figure): bisphenol A epoxide content, bisphenol F epoxide content, tetraglycidyl ether content of tetraphenylethane, glycidyloxyphenylenemethylene group content (of phenol novolak resins), glycidyloxytolylenemethylene group content (of cresol novolak resins), epoxy group content, naphthyldiglycidyl group content, catalyst contents, for example tetraalkylammonium salts, lithium bromide, choline chloride, imidazoles, triglycidyl cyanurate groups, equivalents ratios such as epoxide to amine, epoxide to carboxyl group ratios.

Descriptors particularly suitable for polyurethanes are (as equivalent weight or weight percentage figure): urethane group content, urea group content, biuret group content, isocyanurate group content, allophanate group content, ester group content, ether group content, carbonate group content, carboxyl group content, carbonyl group content, sulfonic acid group content, OH group content, NCO group content (for example for prepolymers), H donor group content (as equivalent weight), H acceptor group content (as equivalent weight), node point density (as equivalent weight), hard segment content (as proportion by weight, defined as the proportion of all urethanized diols),

proportions by weight of customary isocyanate components, for example butane 1,4-diisocyanate, pentane 1,5-diisocyanate, hexane 1,6-diisocyanate (hexamethylene diisocyanate, HDI), 2,2,4-trimethylhexamethylene diisocyanate and/or 2,4,4-trimethylhexamethylene diisocyanate (TMDI), isophorone diisocyanate (IPDI), bis(4,4′-isocyanatocyclohexyl)methane and/or bis(2′,4-isocyanatocyclohexyl)methane, tolylene 2,4- and/or 2,6-diisocyanate (TDI), naphthylene 1,5-diisocyanate (NDI), diphenylmethane 2,4′- and/or 4,4′-diisocyanate (MDI), 1,3-bis(isocyanatomethyl)benzene (XDI) and/or the analogous 1,4 isomer,

proportions by weight of customary alcohol components or ether precursors, for example ethylene glycol, propylene glycol, also ethylene oxide, propylene oxide, butanediol, neopentyl glycol, hexanediol, trimethylolpropane, glycerol, pentaerythritol, sugars and derivatives thereof (e.g. sorbitol),

proportions by weight of customary acids in polyesters, for example adipic acid, terephthalic acid, isophthalic acid, phthalic acid/anhydride, trimellitic acid/anhydride, succinic acid, suberic acid, sebacic acid, decanedicarboxylic acid,

proportions by weight of customary monomers in polyacrylate units, for example methyl acrylate, methyl methacrylate, ethyl acrylate, ethyl methacrylate, ethoxyethyl acrylate, ethoxyethyl methacrylate, n-butyl acrylate, n-butyl methacrylate, tert-butyl acrylate, tert-butyl methacrylate, hexyl acrylate, hexyl methacrylate, 2-ethylhexyl acrylate, 2-ethylhexyl methacrylate, butoxyethyl acrylate, butoxyethyl methacrylate, lauryl acrylate, lauryl methacrylate, isobornyl acrylate, isobornyl methacrylate, phenyl acrylate, phenyl methacrylate, phenoxyethyl acrylate, phenoxyethyl methacrylate, phenoxyethoxyethyl acrylate, phenoxyethoxyethyl methacrylate, bisphenol A diacrylate, bisphenol A dimethacrylate, 2-hydroxyethyl (meth)acrylate, 2-hydroxypropyl (meth)acrylate, 4-hydroxybutyl (meth)acrylate, stearyl acrylates, all of which also find use in other acrylate paint systems, for example polyacrylate dispersions,

equivalents ratios of isocyanate to alcohol, isocyanate to amine and isocyanate to the sum total of the isocyanate-reactive groups.

Suitable descriptors for radiation-curing systems are especially the proportions by weight of customary higher-functionality reactive diluents, for example: hexanediol diacrylate, butanediol diacrylate, di- and tripropylene glycol diacrylate, bisphenol A diacrylate, trimethylolpropane triacrylate, pentaerythritol triacrylate, trimethylolpropane tetraacrylate, dipentaerythritol penta- and hexaacrylate, benzyl methacrylate, isodecyl methacrylate, ethylene glycol diacrylate, bisphenol A epoxyacrylate, phosphate methacrylate, carboxyethyl acrylate.

As descriptors for hybrid systems that combine structural elements of the described material technologies of epoxy resins, phenolic resins, vulcanizable rubber mixtures, unsaturated polyester/styrene mixtures, silicones or polyurethanes, or occur collectively in product combinations, it is of course also possible to combine the abovementioned descriptors: for example, in isocyanate-modified epoxy systems, as well as the descriptors particularly suitable for epoxides, it is also possible to use those descriptors suitable for polyurethanes and utilize them collectively in the method proposed.

In addition, illustrative behavior properties that are typically part of a target profile of requirements in paints and adhesives applications and hence are employed as test feature values Pᵢ of behavior properties are now to be specified. A distinction is made here between paint properties such as flashpoint, pot life, drying time, viscosity, surface tension, abrasion resistance, results from the capillary method or the bubble pressure tensiometer, surface properties of the hardened/dried paint, for example gloss, color, hardness, scratch resistance, notching, flexibility, wetting faults such as dewetting, crater formation, fish eyes, Bénard cells, orange skin, surface roughness, sliding properties, effect surface properties such as structure paints, hammer blow effects,

bulk properties of the hardened/dried paint, film thickness, hiding power, for example Hansen parameters, viscosity of the composition (depending on temperature, shear rate), structural viscosity, thixotropy, dilatancy, rheopexy, mechanical properties such as elasticity and extensibility (for example linear elastic deformation, plastic deformation, yield stress, yield point, breaking stress, elongation at break), but also complete system tests such as water vapor resistance, water resistance, solvent resistance, corrosion resistance, weatherability, biodegradation by bacteria, infestation by fungi, algae or barnacles,

adhesion properties of the hardened/dried paint, for example adhesion force in a peel test on the loss of adhesion, the result of a crosscut test, and adhesion stability.

In the case of adhesives, tensile stress, pull tests, shear stress, flex testing, flexural testing, impact resistance, fracture characteristics such as cohesion or adhesion fracture, fracture type testing, surface tension, thermal and moisture stability, instant adhesion, long-term adhesion and curing time are suitable behavior properties.

The method proposed is particularly suitable for the development of product compositions and product distributions thereof for thermoplastics, thermosets, 2-component reactive materials, paints, adhesives, casting compounds, foams, and with particular preference for paints and adhesives. In particular, it is also suitable for epoxy systems, phenolic resin systems, vulcanizable rubber mixtures, unsaturated polyester/styrene mixtures, silicone systems or polyurethane systems, suitable systems being more preferably epoxy systems, silicone systems or polyurethane systems and most preferably polyurethane systems.

The proposed method of producing a mixed chemical product from a product composition is characterized in that the product composition has been ascertained by the proposed method of ascertaining a product composition for a mixed chemical product.

The proposed mixed chemical product is characterized in that the mixed chemical product has been produced by the proposed method of producing a mixed chemical product.

Preferred embodiments and variants of the proposed method of producing a mixed chemical product and of the mixed chemical product proposed corresponding to preferred embodiment and variants of the proposed method of ascertaining a product composition, and vice versa.

Further details, features, configurations, aims and advantages of the present invention are elucidated hereinafter with reference to the drawing. The drawing shows:

FIG. 1 by way of example, a strong positive correlation of the urea content as descriptor with the modulus of elasticity as behavior property,

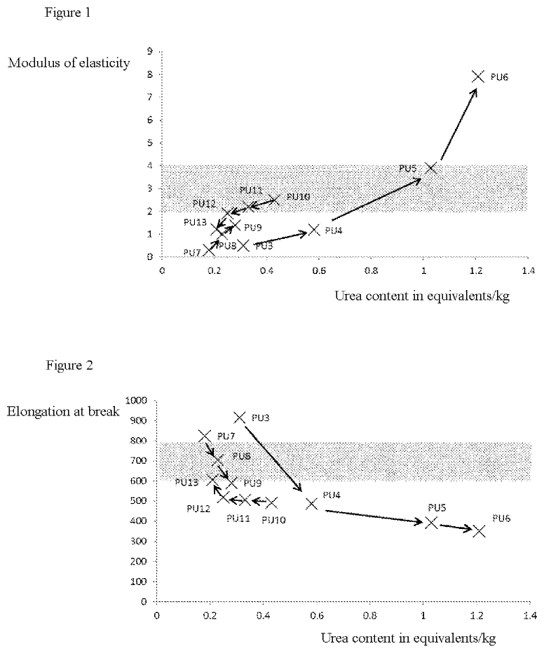

FIG. 2 by way of example, a negative correlation of elongation at break as behavior property associated with the correlation of FIG. 1,

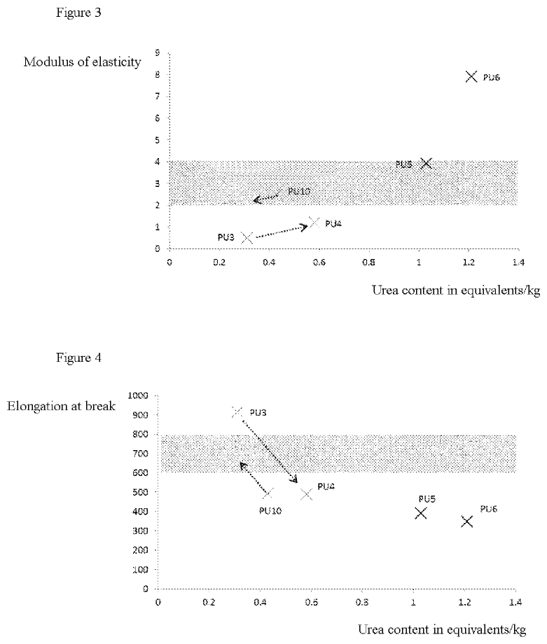

FIG. 3 by way of example, the derivative of the variation in modulus of elasticity as behavior property between two product compositions and the prediction of the variation in modulus of elasticity in the case of a varied product composition,

FIG. 4 by way of example, the derivative of the variation in elongation at break as behavior property between the two product compositions of FIG. 3 and the prediction of the variation in elongation at break in the case of the varied product composition of FIG. 3, and

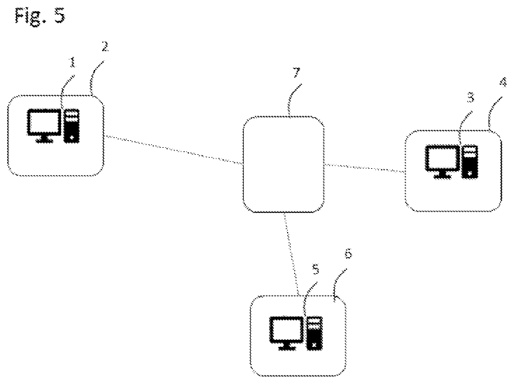

FIG. 5 three computer systems for execution of a working example of the proposed method of ascertaining a product composition for a mixed chemical product.

The invention is also elucidated hereinafter with reference to tables. The tables show: Table A: various polyurethanes as product compositions with product distributions, taken from W. Panwiriyarat et al., J. Polym. Environ. 21, 807-815 (2013), Table 1, top of p. 809. The molar ratios of the four composition constituents are specified here: IPDI: isophorone diisocyanate with a molar mass of 220; PCL: polycaprolactonediol prepared from ethylene glycol and caprolactone with a molar mass of 530; HTNR: an unsaturated rubber diol having a molar mass of 1700 with an average of 23.6 double bonds and BDO: butanediol with a molar mass of 90.

Table B shows the behavior properties of the products from table A, taken from W. Panwiriyarat et al., J. Polym. Environ. 21, 807-815 (2013), Table 2, p. 811 with modulus of elasticity in MPa (“YoungMod”), breaking stress in MPa (“TensileStr”), elongation at break in percent (“EaB”), tear strength in N/mm² (“TearStr”) and Shore A hardness—unitless—(“Shore A”)

Table 1a: For polyurethanes 3 to 14 the descriptors: hard segment content in percent by weight (“HardSeg”), urea content in equivalents/kg (“Urea”), urethane content in equivalents/kg (“Urethane”), ester content in equivalents/kg (“Ester”), double bond content in equivalents/kg (“Doublebond”), butanediol content in equivalents/kg (“BDO”), and the performance properties: Pᵢ: modulus of elasticity in MPa (“YoungMod”), breaking stress in MPa (“TensileStr”), elongation at break in percent (“EaB”), tear strength in N/mm² (“TearStr”) and Shore A hardness—unitless—(“Shore A”).

For the calculation of the hard segment content, the proportions by weight of all diisocyanates (IPDI here) and diols (butanediol here) were based on/calculated on the basis of the total weight and reported in percent.

For the calculation of the urea content, the isocyanate excess of the isocyanate groups that have not reacted with alcohol groups was calculated. These react with ambient water to give carbamic acid and with elimination of carbon dioxide to give amine that reacts rapidly with a further isocyanate to give urea. Thus, two excess isocyanate groups give rise to one urea group. The molar amount of urea groups is then based on/calculated on the basis of one kilogram of total product.

The calculation of the urethane content is made here from the alcohol groups present in deficiency. Each reacts to give a urethane group, and so the molar amount of the alcohol groups per kg of total product gives the urethane content in equivalents/kg.

The calculation of the ester content is made via the ester groups present in the polycaprolactone (“PCL”) of 2.05 ester groups per equivalent of polycaprolactone of 265 g. Thus, the ester content is calculated from the amount of ester equivalents in the amount of the polycaprolactone used, based on one kilogram of total product.

The calculation of the double bond content is made via the double bonds present in the unsaturated polybutadienediol (“HTNR”) of 23.6 double bond groups per molar mass of 1700 g/mol of HTNR, at an equivalent weight of 850 g. Thus, the double bond content is calculated from the amount of double bond equivalents in the amount of the HTNR used, based on one kilogram of total product.

The calculation of the butanediol content is made via the amount of butanediol (“BDO”) with an equivalent weight of 45 g, based on one kilogram of total product.

Table 2a: Analogous to table 1a, except that all values have been scaled on a scale from 0 to 1 in which the maximum value—feature value or test feature value—in each column (i.e. of each descriptor and each behavior property) has been determined and each individual value has been divided by this maximum value.

Table 3a: Analogous to table 1a, except that all values have first been squared and then been scaled on a scale from 0 to 1 in which the maximum value in each column (i.e. of each squared descriptor and each squared behavior property) has been determined and each individual squared feature value or test feature value has been divided by this maximum squared value.

Table 4a: Analogous to table 1a: The feature values have been retained and the decadic logarithm has been calculated from the test feature values.

Table 5a: Analogous to table 1a; in this case, the arithmetic average and the variance of the feature values of a descriptor and of the test feature values of a behavior property have first been determined. The arithmetic average is the sum total of all individual values divided by the number of individual values. The variance is calculated from a sum total over all squares of the differences of individual values minus the arithmetic average thereof, and the sum is then divided by the number of individual values. Table 5a gives results from the quotient of individual values minus arithmetic average divided by the square root of the variance (student distribution).

Table 5a-2: As table 5a, except with generically named descriptors M1-M6 and behavior properties P1-P5.

TABLE A

| | IPDI | PCL | HTNR | BDO |
|---|---|---|---|---|
| Polyurethane 3 | 1.25 | 1.00 | 0.00 | 0.00 |
| Polyurethane 4 | 1.50 | 1.00 | 0.00 | 0.00 |
| Polyurethane 5 | 2.00 | 1.00 | 0.00 | 0.00 |
| Polyurethane 6 | 2.25 | 1.00 | 0.00 | 0.00 |
| Polyurethane 7 | 1.25 | 0.50 | 0.50 | 0.00 |
| Polyurethane 8 | 1.25 | 0.35 | 0.35 | 0.30 |
| Polyurethane 9 | 1.25 | 0.25 | 0.25 | 0.50 |
| Polyurethane 10 | 1.25 | 0.50 | 0.00 | 0.50 |
| Polyurethane 11 | 1.25 | 0.35 | 0.15 | 0.50 |
| Polyurethane 12 | 1.25 | 0.15 | 0.35 | 0.50 |
| Polyurethane 13 | 1.25 | 0.00 | 0.50 | 0.50 |

TABLE B

| | Young | | | | |
|---|---|---|---|---|---|
| | Mod | TensileStr | EaB | TearStr | Shore A |
| Polyurethane 3 | 0.5 | 18.9 | 916 | 16.9 | 42 |
| Polyurethane 4 | 1.2 | 22.6 | 488 | 22.8 | 42 |
| Polyurethane 5 | 3.9 | 54.5 | 392 | 56.2 | 45 |
| Polyurethane 6 | 7.9 | 53.6 | 351 | 109.1 | 48 |
| Polyurethane 7 | 0.3 | 3.5 | 824 | 7.5 | 21 |
| Polyurethane 8 | 1.0 | 14.1 | 703 | 13.8 | 32 |
| Polyurethane 9 | 1.4 | 14.9 | 590 | 38.6 | 47 |
| Polyurethane 10 | 2.5 | 21.7 | 493 | 65.4 | 57 |
| Polyurethane 11 | 2.2 | 24.3 | 506 | 38.3 | 56 |
| Polyurethane 12 | 1.9 | 14.2 | 518 | 30.8 | 51 |
| Polyurethane 13 | 1.2 | 9.1 | 605 | 20.9 | 40 |

TABLE 1a

| Name | HardSeg | Urea | Urethane | Ester | Doublebond | BDO | YoungMod | TensileStr | EaB | TearStr | ShoreA |
|---|---|---|---|---|---|---|---|---|---|---|---|
| Polyurethane 3 | 34.37 | 0.31 | 2.48 | 5.08 | 0.00 | 0.00 | 0.50 | 18.90 | 916.00 | 16.90 | 42.00 |
| Polyurethane 4 | 38.59 | 0.58 | 2.32 | 4.75 | 0.00 | 0.00 | 1.20 | 22.60 | 488.00 | 22.80 | 42.00 |
| Polyurethane 5 | 45.59 | 1.03 | 2.05 | 4 21 | 0.00 | 0.00 | 3.90 | 54.60 | 392.00 | 56.20 | 45.00 |
| Polyurethane 6 | 48.52 | 1.21 | 1.94 | 3.98 | 0.00 | 0.00 | 7.90 | 53.60 | 351.00 | 109.10 | 48.00 |
| Polyurethane 7 | 19.93 | 0.18 | 1.44 | 1.47 | 16.88 | 0.00 | 0.30 | 3.50 | 824.00 | 7.50 | 21.00 |
| Polyurethane 8 | 28.07 | 0.23 | 1.84 | 1.32 | 15.16 | 0.55 | 1.00 | 14.10 | 703.00 | 13.80 | 32.00 |
| Polyurethane 9 | 36.65 | 0.28 | 2.27 | 1.17 | 13.35 | 1.14 | 1.40 | 14.90 | 590.00 | 38.60 | 47.00 |
| Polyurethane 10 | 54.90 | 0.43 | 3.40 | 3.49 | 0.00 | 1.70 | 2.50 | 21.70 | 493.00 | 65.40 | 57.00 |
| Polyurethane 11 | 42.27 | 0.33 | 2.62 | 1.88 | 9.24 | 1.31 | 2.20 | 24.30 | 506.00 | 38.30 | 56.00 |
| Polyurethane 12 | 32.35 | 0.25 | 2.01 | 0.62 | 16.50 | 1.00 | 1.90 | 14.20 | 518.00 | 30. [symbol] | 51.00 |
| Polyurethane 13 | 27.51 | 0.21 | 1.71 | 0.00 | 20.04 | 0. [symbol] | 1.20 | 9.10 | 605.00 | 20.00 | 40.00 |

[symbol]

indicates data missing or illegible when filed

TABLE 2a

| Name | HardSeg | Urea | Urethane | Ester | Doublebond | BDO | YoungMod | TensileStr | EaB | TearStr | ShoreA |
|---|---|---|---|---|---|---|---|---|---|---|---|
| Polyurethane 3 | 0.6260 | 0.2550 | 0.7277 | 1.0000 | 0.0000 | 0.0000 | 0.0633 | 0.3462 | 1 0000 | 0.1549 | 0.7368 |
| Polyurethane 4 | 0.7029 | 0.4773 | 0.6807 | 0.9358 | 0.0000 | 0.0000 | 0.1519 | 0.4139 | 0.5328 | 0.2090 | 0.7368 |
| Polyurethane 5 | 0.8304 | 0.8460 | 0.6031 | 0.8290 | 0.0000 | 0.0000 | 0.4937 | 1.0000 | 0.4279 | 0.5151 | 0.7895 |
| Polyurethane 6 | 0.8838 | 1.0000 | 0.5708 | 0.7845 | 0.0000 | 0.0000 | 1.0000 | 0.9817 | 0.3832 | 1.0000 | 0.8421 |
| Polyurethane 7 | 0.3630 | 0.1479 | 0.4219 | 0.2899 | 0.8423 | 0.0000 | 0.0380 | 0.0641 | 0.8996 | 0.0687 | 0.3684 |
| Polyurethane 8 | 0.5113 | 0.1898 | 0.5414 | 0.2606 | 0.7 [symbol] | 0.3235 | 0.1266 | 0.2582 | 0.7675 | 0.1265 | 0.5614 |
| Polyurethane 9 | 0.6676 | 0.2340 | 0.6677 | 0.2295 | 0.6662 | 0.6706 | 0.1772 | 0.2729 | 0.6441 | 0.3538 | 0.8246 |
| Polyurethane 10 | 1.0000 | 0.3505 | 1.0000 | 0.6872 | 0.0000 | 1.0000 | 0.3165 | 0.3974 | 0.5382 | 0.5995 | 1.0000 |
| Polyurethane 11 | 0.7699 | 0.2699 | 0.7700 | 0.3705 | 0.4610 | 0.7706 | 0.2785 | 0.4451 | 0 5524 | 0.3511 | 0.9825 |
| Polyurethane 12 | 0.5893 | 0.2065 | 0.5893 | 0.1215 | 0.8234 | 0.5882 | 0.2405 | 0.2601 | 0.5655 | 0.2823 | 0.8947 |
| Polyurethane 13 | 0.5011 | 0.1756 | 0.5012 | 0.0000 | 1.0000 | 0.5000 | 0.1519 | 0.1667 | 0.6605 | 0.1833 | 0.7018 |

[symbol]

indicates data missing or illegible when filed

TABLE 3a

| Name | HardSeg | Urea | Urethane | Ester | Doublebond | BDO | YoungMod | TensileStr | EaB | TearStr | ShoreA |
|---|---|---|---|---|---|---|---|---|---|---|---|
| Polyurethane 3 | 0.3919 | 0.0650 | 0.5295 | 1.0000 | 0.0000 | 0.0000 | 0.0040 | 0.1198 | 1.0000 | 0.0240 | 0.5429 |
| Polyurethane 4 | 0.4941 | 0.2278 | 0.4633 | 0.8757 | 0.0000 | 0.0000 | 0.0231 | 0.1713 | 0.2838 | 0.0437 | 0.5429 |
| Polyurethane 5 | 0.6896 | 0.7157 | 0.3637 | 0.6873 | 0.0000 | 0.0000 | 0.2437 | 1.0000 | 0.1831 | 0.2654 | 0.6233 |
| Polyurethane 6 | 0.7811 | 1.0000 | 0.3258 | 0.6155 | 0.0000 | 0.0000 | 1.0000 | 0.9637 | 0.1468 | 1.0000 | 0.7091 |
| Polyurethane 7 | 0.1318 | 0.0219 | 0.1780 | 0.0841 | 0.7095 | 0.0000 | 0.0014 | 0.0041 | 0.8092 | 0.0047 | 0.1357 |
| Polyurethane 8 | 0.2614 | 0.0360 | 0.2931 | 0.0679 | 0.5723 | 0.1047 | 0.0160 | 0.0667 | 0.5890 | 0.0160 | 0.3152 |
| Polyurethane 9 | 0.4457 | 0.0548 | 0.4459 | 0.0527 | 0.4438 | 0.4497 | 0.0314 | 0.0745 | 0.4149 | 0.1252 | 0.6799 |
| Polyurethane 10 | 1.0000 | 0.1228 | 1.0000 | 0.4723 | 0.0000 | 1.0000 | 0.1001 | 0.1580 | 0.2897 | 0.3593 | 1.0000 |
| Polyurethane 11 | 0.5928 | 0.0728 | 0.5929 | 0.1373 | 0.2125 | 0.5938 | 0.0776 | 0.1981 | 0.3051 | 0.1232 | 0.9652 |
| Polyurethane 12 | 0.3472 | 0.0426 | 0.3473 | 0.0148 | 0.6779 | 0.3460 | 0.0578 | 0.0676 | 0.3198 | 0.0797 | 0.8006 |
| Polyurethane 13 | 0.2511 | 0.0308 | 0.2512 | 0.0000 | 1.0000 | 0.2500 | 0.0231 | 0.0278 | 0.4362 | 0.0336 | 0.4925 |

TABLE 4a

| Name | HardSeg | Urea | Urethane | Ester | Doublebond | BDO | YoungMod TensileStr | | EaB | TearStr | ShoreA |
|---|---|---|---|---|---|---|---|---|---|---|---|
| Polyurethane 3 | 34.37 | 0.31 | 2.48 | 5.08 | 0.00 | 0.00 | −0.30 | 1.28 | 2.96 | 1.23 | 1.62 |
| Polyurethane 4 | 38.69 | 0.58 | 2.32 | 4.75 | 0.00 | 0.00 | 0.08 | 1.35 | 2. [symbol] | 1.3 [symbol] | 1.62 |
| Polyurethane 5 | 45.69 | 1.03 | 2 05 | 4.21 | 0.00 | 0.00 | 0.59 | 1.74 | 2. [symbol] | 1.75 | 1.65 |
| Polyurethane 6 | 48.62 | 1.21 | 1.94 | 3.98 | 0.00 | 0.00 | 0.90 | 1.73 | 2. [symbol] | 2.04 | 1.68 |
| Polyurethane 7 | 19.93 | 0.18 | 1.44 | 1.47 | 16.88 | 0.00 | −0.52 | 0.54 | 2.92 | 0.88 | 1.32 |
| Polyurethane 8 | 28.07 | 0.23 | 1.84 | 1.32 | 16.16 | 0.55 | 0.00 | 1.15 | 2.65 | 1.14 | 1.61 |
| Polyurethane 9 | 36.65 | 0.28 | 2.27 | 1.17 | 13.35 | 1.14 | 0.15 | 1.17 | 2.77 | 1.59 | 1.67 |
| Polyurethane 10 | 54.90 | 0.43 | 3.40 | 3.49 | 0.00 | 1.70 | 0.40 | 1.34 | 2.69 | 1.82 | 1.76 |
| Polyurethane 11 | 42.27 | 0.33 | 2.62 | 1.88 | 9.24 | 1.31 | 0.34 | 1.39 | 2.70 | 1.58 | 1.75 |
| Polyurethane 12 | 32.35 | 0.25 | 2.01 | 0.62 | 16.50 | 1.00 | 0.28 | 1.15 | 2.71 | 1.49 | 1.71 |
| Polyurethane 13 | 27.61 | 0.21 | 1.71 | 0.00 | 20.04 | 0.85 | 0.08 | 0.96 | 2.78 | 1.30 | 1.60 |

[symbol]

indicates data missing or illegible when filed

TABLE 5a

| Name | HardSeg | Urea | Urethane | Ester | Doublebond | BDO | YoungMod | TensileStr | EaB | TearStr | ShoreA |
|---|---|---|---|---|---|---|---|---|---|---|---|
| Polyurethane 3 | −0.2859 | −0.4478 | 0.5707 | 1.4816 | −1.0415 | −0.9837 | −0.8212 | −0.2506 | 2.0265 | −0.7546 | −0.1740 |
| Polyurethane 4 | 0.1467 | 0.3648 | 0.2536 | 1.2911 | −1.0415 | −0.9837 | −0.4794 | −0.0167 | −0.5591 | −0.5448 | −0.1740 |
| Polyurethane 5 | 0.8643 | 1.7129 | 0.2695 | 0.9742 | −1.0415 | −0.9837 | 0.8390 | 2.0069 | −1.1390 | 0.6424 | 0.1282 |
| Polyurethane 6 | 1.1646 | 2.2761 | −0.4874 | 0.8421 | −1.0415 | −0.9837 | 2.7921 | 1.9437 | −1.3867 | 2.5229 | 0.4304 |
| Polyurethane 7 | −1.7662 | −0.8396 | −1.4920 | −0.6257 | 1.0797 | −0.9837 | −0.9189 | −1.2245 | 1.4707 | −1.0887 | −2.2892 |
| Polyurethane 8 | −0.9317 | −0.6863 | −0.6856 | −0.7128 | 0.8636 | −0.0751 | −0.5771 | −0.5542 | 0.7397 | −0.8648 | −1.1813 |
| Polyurethane 9 | −0.0522 | −0.5246 | 0.1664 | −0.8052 | 0.6361 | 0.8996 | −0.3818 | −0.5036 | 0.0571 | 0.0168 | 0.3297 |
| Polyurethane 10 | 1.8186 | −0.0987 | 2.4075 | 0.5533 | −1.0415 | 1.8247 | 0.1554 | −0.0736 | −0.5289 | 0.9695 | 1.3369 |
| Polyurethane 11 | 0.5239 | −0.3936 | 0.8560 | −0.3867 | 0.1195 | 1.1805 | 0.0089 | 0.0908 | −0.4503 | 0.0061 | 1.2362 |
| Polyurethane 12 | −0.4930 | −0.6252 | −0.3626 | −1.1257 | 1.0319 | 0.6683 | −0.1376 | −0.5479 | −0.3778 | −0.2605 | 0.7326 |
| Polyurethane 13 | −0.9891 | −0.7381 | −0.9570 | −1.4862 | 1.4768 | 0.4205 | −0.4794 | −0.8704 | 0.1477 | −0. [symbol] 444 | −0.3754 |

[symbol]

indicates data missing or illegible when filed

TABLE 5a-2

| Name | M1 | M2 | M3 | M4 | M5 | M6 | P1 | P2 | P3 | P4 | P5 |
|---|---|---|---|---|---|---|---|---|---|---|---|
| Polyurethane 3 | −0.2859 | −0.4478 | 0.5707 | 1.4816 | −1.0415 | −0.9837 | −0.8212 | −0.2506 | 2.0265 | −0.7546 | −0.1740 |
| Polyurethane 4 | 0.1467 | 0.3648 | 0.2536 | 1.2911 | −1.0415 | −0.9837 | 0.4794 | −0.0167 | −0.5591 | −0.5448 | −0.1740 |
| Polyurethane 5 | 0.8643 | 1.7129 | −0.2695 | 0.9742 | −1.0415 | −0.9837 | 0.8390 | 2.0069 | −1.1390 | 0.6424 | 0.1282 |
| Polyurethane 6 | 1.1646 | 2.2761 | −0.4874 | 0.8421 | −1.0415 | −0.9837 | 2.7921 | 1.9437 | −1.3867 | 2.5229 | 0.4304 |
| Polyurethane 7 | −1.7662 | −0.8396 | −1.4920 | −0.6257 | 1.0797 | −0.9837 | −0.9189 | −1.2245 | 1.4707 | −1.0887 | −2.2892 |
| Polyurethane 8 | −0.9317 | −0.6863 | −0.6856 | −0.7128 | 0.8636 | −0.0751 | −0.5771 | −0.5542 | 0.7397 | −0.8648 | −1.1813 |
| Polyurethane 9 | −0.0522 | −0.5246 | 0.1664 | −0.8052 | 0.6361 | 0.8996 | −0.3818 | −0.5036 | 0.0571 | 0.0168 | 0.3297 |
| Polyurethane 10 | 1.8186 | −0.0987 | 2.4075 | 0.5533 | −1.0415 | 1.8247 | 0.1554 | −0.0736 | −0.5289 | 0.9695 | 1.3369 |
| Polyurethane 11 | 0.5239 | −0.3936 | 0.8560 | −0.3867 | 0.1195 | 1.1805 | 0.0089 | 0.0908 | −0.4503 | 0.0061 | 1.2362 |
| Polyurethane 12 | −0.4930 | −0.6252 | −0.3626 | −1.1257 | 1.0319 | 0.6683 | −0.1376 | −0.5479 | −0.3778 | −0.2605 | 0.7326 |
| Polyurethane 13 | −0.9891 | −0.7381 | −0.9570 | −1.4862 | 1.4768 | 0.4205 | −0.4794 | −0.8704 | 0.1477 | −0.6444 | −0.3754 |

Table 1b: Table 1a was read into R in csv file format. The following R version was used: R version 3.4.3 (Nov. 30, 2017)—“Kite-Eating Tree”, Copyright © 2017 The R Foundation for Statistical Computing, Platform: x86_64-w64-mingw32/x64 (64-bit). Subsequently, the “cor” command was used to calculate the Pearson correlation matrix-correlation matrix hereinafter.

For 11 formulations, with df (degree of freedom)=11-2=9, a critical Pearson correlation value of 0.602 is found. Correlation values>0.602 show an upward arrow (positive correlation); correlation values<−0.602 show a downward arrow (negative correlation); all intermediate values show no (statistical) correlation and hence a horizontal arrow.

Table 2b: Correlation matrix analogous to table 1b, except that table 2a was read in. The table is identical to table 1b.

Table 3b: Correlation matrix analogous to table 1b, except that table 3a was read in.

Table 4b: Correlation matrix analogous to table 1b, except that table 4a was read in.

Table 5b: Correlation matrix analogous to table 1b, except that table 5a was read in. The table is identical to table 1b.

Table 5b-2: As table 5b, except with generically named descriptors M1-M6 and test features P1-P5.

Tables 1b, 2b and 5b show correlation results that are the same except for rounding errors.

It has thus been shown that, in selected cases of the mapping of all feature values of all product compositions and of the mapping of all test feature values by a bijective, constant, strictly monotonous and selectively normalizing function, identical results can occur: specifically in table 1a->2a with a scale (=normalization) and in table 1a->5a with Student's distribution.

If table 1b is compared with 3b, slight differences are seen in the correlation coefficients, which are similar in terms of sign and magnitude, but in individual cases lead to a different assessment in terms of the criterion of the critical Pearson correlation value. (cf., for example: c(Pearson)_{Urea-Eab}=−0.694 (in table 1b) vs. c(Pearson)_{Urea-Eab}=−0.556 (in table 3b)). This can be explained by the better correlation by a linear function compared to a quadratic function.

It follows that the better Pearson correlation value in table 1b suggests that a more suitable bijective mapping maps the experimental data better than in table 3b.

Reference is made to an analogous example with c(Pearson)_{Ester-TensileStr}=0.600 (table 1b), c(Pearson)_{Ester-TensileStr}=0.457 (table 3b), c(Pearson)_{Ester-TensileStr}=0.619 (table 4b). It is found here that in table 4b with the decadic logarithm as bijective, constant, strictly monotonous function better results are found.

Thus, the positive and negative correlations from tables 1b-5b are considered collectively and the maximum correlation value (for positive values) or the minimum correlation value (for negative values) is chosen in each case and compared with the critical correlation value. In this way, an overall view is obtained (in table 3 here), and it becomes clear that various bijective, constant, strictly monotonous and selectively normalizing mappings or functions should be examined. This may be assisted by graph representation of the data. In qualitative terms, a good overview of which descriptors have a positive or negative correlation with which behavior properties is thus obtained.

TABLE 1b

| | HardSeg | | Urea | | Urethane | | Ester | | Doublebond | | BDO | |
|---|---|---|---|---|---|---|---|---|---|---|---|---|
| HardSeg | ↑ | 1.000 | ↑ | 0.650 | ↑ | 0.751 | → | 0.566 | ↓ | −0.774 | → | 0.281 |
| Urea | ↑ | 0.650 | ↑ | 1.000 | → | 0.025 | ↑ | 0.614 | ↓ | −0.693 | → | −0.43 [symbol] |
| Urethane | ↑ | 0.751 | → | 0.025 | ↑ | 1.000 | → | 0.420 | → | −0.577 | → | 0.578 |
| Ester | → | 0.565 | ↑ | 0.614 | → | 0.420 | ↑ | 1.000 | ↓ | −0. [symbol] 60 | → | −0.490 |
| Doublebond | ↓ | −0.774 | ↓ | −0.593 | → | −0.577 | ↓ | −0.960 | ↑ | 1.000 | → | 0.281 |
| BDO | → | 0.281 | → | 0.436 | → | 0.578 | → | −0.490 | → | 0.281 | ↑ | 1.000 |
| YoungMod | ↑ | 0.650 | ↑ | 0.887 | → | 0.035 | → | 0.325 | → | −0.470 | → | −0.153 |
| TensileStr | ↑ | 0.711 | ↑ | 0.864 | → | 0.125 | → | 0.600 | ↓ | −0.702 | → | −0.325 |
| EaB | ↓ | −0.701 | ↓ | −0.694 | → | −0.212 | → | −0.127 | → | 0.335 | → | −0.191 |
| TearStr | ↑ | 0.808 | ↑ | 0.520 | → | 0.297 | → | 0.363 | → | −0.553 | → | 0.059 |
| ShoreA | ↑ | 0.805 | → | 0.272 | ↑ | 0.751 | → | 0.183 | → | −0.413 | ↑ | 0.605 |

| | YoungMod | | TensileStr | | EaB | | TearStr | | ShoreA | |
|---|---|---|---|---|---|---|---|---|---|---|
| HardSeg | ↑ | 0.650 | ↑ | 0.711 | ↓ | −0.701 | ↑ | 0.808 | ↑ | 0.805 |
| Urea | ↑ | 0.887 | ↑ | 0.964 | ↓ | −0.694 | ↑ | 0.820 | → | 0.272 |
| Urethane | → | 0.035 | → | 0.126 | → | −0.212 | → | 0.2 [symbol] 7 | ↑ | 0.751 |
| Ester | → | 0.325 | → | 0.600 | → | −0.127 | → | 0.363 | → | 0.183 |
| Doublebond | → | −0.470 | ↓ | −0.702 | → | 0.335 | → | −0.553 | → | −0.413 |
| BDO | → | −0.153 | → | −0.326 | → | −0.191 | → | 0.059 | ↑ | 0.605 |
| YoungMod | ↑ | 1.000 | ↑ | 0.859 | ↓ | −0.739 | ↑ | 0.850 | → | 0.408 |
| TensileStr | ↑ | 0.858 | ↑ | 1.000 | ↓ | −0.695 | ↑ | 0.503 | → | 0.397 |
| EaB | ↓ | −0.739 | ↓ | −0.695 | ↑ | 1.000 | ↓ | −0.746 | ↓ | −0.620 |
| TearStr | ↑ | 0.850 | ↑ | 0.803 | ↓ | −0.746 | ↑ | 1.000 | → | 0.570 |
| ShoreA | → | 0.408 | → | 0.397 | ↓ | −0.620 | → | 0.570 | ↑ | 1.000 |

[symbol]

indicates data missing or illegible when filed

TABLE 2b

| | HardSeg | | Urea | | Urethane | | Ester | | Doublebond | | BDO | |
|---|---|---|---|---|---|---|---|---|---|---|---|---|
| HardSeg | ↑ | 1.000 | ↑ | 0.647 | ↑ | 0.754 | → | 0.566 | ↓ | −0.774 | → | 0.281 |
| Urea | ↑ | 0.647 | ↑ | 1.000 | → | 0.024 | ↑ | 0.612 | ↓ | −0.689 | → | −0.437 |
| Urethane | ↑ | 0.754 | → | 0.024 | ↑ | 1.000 | → | 0.420 | → | −0.578 | → | 0.578 |
| Ester | → | 0.566 | ↑ | 0.612 | → | 0.420 | ↑ | 1.000 | ↓ | −0.960 | → | −0.490 |
| Doublebond | ↓ | −0.774 | ↓ | −0.689 | → | −0.575 | ↓ | −0.960 | ↑ | 1.000 | → | 0.281 |
| BDO | → | 0.201 | → | −0.437 | → | 0.575 | → | −0.490 | → | 0.281 | ↑ | 1.000 |
| YoungMod | ↑ | 0.650 | ↑ | 0.889 | → | 0.035 | → | 0.325 | → | −0.470 | → | −0.15 [symbol] |
| TensileStr | ↑ | 0.711 | ↑ | 0.963 | → | 0.129 | → | 0.600 | ↓ | −0.702 | → | −0.326 |
| EaB | ↓ | −0.701 | ↓ | −0.694 | → | −0.214 | → | −0.128 | → | 0.336 | → | −0.191 |
| TearStr | ↑ | 0.808 | ↑ | 0.820 | → | 0.300 | → | 0.363 | → | −0.553 | → | 0.059 |
| ShoreA | ↑ | 0.805 | → | 0.270 | ↑ | 0.751 | → | 0.163 | → | −0.413 | ↑ | 0.606 |

| | YoungMod | | TensileStr | | EaB | | TearStr | | ShoreA | |
|---|---|---|---|---|---|---|---|---|---|---|
| HardSeg | ↑ | 0.650 | ↑ | 0.711 | ↓ | −0.701 | ↑ | 0.508 | ↑ | 0.805 |
| Urea | ↑ | 0.889 | ↑ | 0.863 | ↓ | −0.694 | ↑ | 0.520 | → | 0.270 |
| Urethane | → | 0.038 | → | 0.129 | → | −0.214 | → | 0.300 | ↑ | 0.751 |
| Ester | → | 0.325 | → | 0.600 | → | −0.128 | → | 0.363 | → | 0.183 |
| Doublebond | → | −0.470 | ↓ | −0.702 | → | 0.336 | → | −0.553 | → | −0.413 |
| BDO | → | −0.153 | → | −0.326 | → | −0.191 | → | 0.059 | ↑ | 0.606 |
| YoungMod | ↑ | 1.000 | ↑ | 0.859 | ↓ | −0.739 | ↑ | 0.950 | → | 0.406 |
| TensileStr | ↑ | 0.859 | ↑ | 1.000 | ↓ | −0.695 | ↑ | 0.502 | → | 0.397 |
| EaB | ↓ | −0.739 | ↓ | −0.695 | ↑ | 1.000 | ↓ | −0.746 | ↓ | −0.620 |
| TearStr | ↑ | 0.950 | ↑ | 0.802 | ↓ | −0.746 | ↑ | 1.000 | → | 0.570 |
| ShoreA | → | 0.40 [symbol] | → | 0.397 | ↓ | −0.620 | → | 0.570 | ↑ | 1.000 |

[symbol]

indicates data missing or illegible when filed

TABLE 3b

| | HardSeg | | Urea | | Urethane | | Ester | | Doublebond | | BDO | |
|---|---|---|---|---|---|---|---|---|---|---|---|---|
| HardSeg | ↑ | 1.000 | → | 0.552 | ↑ | 0.746 | → | 0.441 | ↓ | −0.759 | → | 0.498 |
| Urea | → | 0.552 | ↑ | 1.000 | → | −0.119 | → | 0.467 | → | −0.540 | → | −0.355 |
| Urethane | ↑ | 0.746 | → | −0.119 | ↑ | 1.000 | → | 0.307 | → | −0.574 | ↑ | 0.784 |
| Ester | → | 0.441 | → | 0.467 | → | 0.307 | ↑ | 1.000 | ↓ | −0.839 | → | −0.334 |
| Doublebond | ↓ | −0.759 | → | −0.540 | → | −0.574 | ↓ | −0.839 | ↑ | 1.000 | → | −0.027 |
| BDO | → | 0.498 | → | −0.355 | ↑ | 0.784 | → | −0.334 | → | −0.027 | ↑ | 1.000 |
| YoungMod | → | 0.504 | ↑ | 0.597 | → | −0.106 | → | 0.267 | → | −0.388 | → | −0.216 |
| TensileStr | → | 0.572 | ↑ | 0.968 | → | −0.077 | → | 0.457 | → | −0.563 | → | −0.309 |
| EaB | ↓ | −0.628 | → | −0. [symbol] | → | −0.190 | → | 0.036 | → | 0.249 | → | −0.292 |
| TearStr | ↑ | 0.676 | ↑ | 0.853 | → | 0.125 | → | 0.274 | → | −0.468 | → | 0.009 |
| ShoreA | ↑ | 0.765 | → | 0.153 | ↑ | 0.740 | → | 0.091 | → | −0.444 | ↑ | 0.731 |

| | YoungMod | | TensileStr | | EaB | | TearStr | | ShoreA | |
|---|---|---|---|---|---|---|---|---|---|---|
| HardSeg | → | 0.504 | → | 0.572 | ↓ | −0.628 | ↑ | 0.676 | ↑ | 0.765 |
| Urea | ↑ | 0.897 | ↑ | 0.968 | ↓ | −0.556 | ↑ | 0.853 | → | 0.153 |
| Urethane | → | −0.105 | → | −0.077 | → | −0.180 | → | 0.125 | ↑ | 0.740 |
| Ester | → | 0.267 | → | 0.457 | → | 0.038 | → | 0.274 | → | 0.091 |
| Doublebond | → | −0.388 | → | −0.563 | → | 0.249 | → | −0.468 | → | −0.444 |
| BDO | → | −0.218 | → | −0.309 | → | −0.292 | → | 0.009 | ↑ | 0.731 |
| YoungMod | ↑ | 1.000 | ↑ | 0.795 | → | −0.488 | ↑ | 0.986 | → | 0.210 |
| TensileStr | ↑ | 0.795 | ↑ | 1.000 | → | −0.553 | ↑ | 0.757 | → | 0.203 |
| EaB | → | −0.485 | → | −0.553 | ↑ | 1.000 | → | −0.546 | → | −0.598 |
| TearStr | ↑ | 0.965 | ↑ | 0.757 | → | −0.545 | ↑ | 1.000 | → | 0.369 |
| ShoreA | → | 0.210 | → | 0.203 | → | −0.586 | → | 0.359 | ↑ | 1.000 |

[symbol]

indicates data missing or illegible when filed

TABLE 4b

| | HardSeg | | Urea | | Urethane | | Ester | | Doublebond | | BDO | |
|---|---|---|---|---|---|---|---|---|---|---|---|---|
| HardSeg | ↑ | 1.000 | → | 0.650 | ↑ | 0.751 | → | 0.566 | ↓ | −0.774 | → | 0.281 |
| Urea | → | 0.650 | ↑ | 1.000 | → | 0.025 | ↑ | 0.614 | ↓ | −0.693 | → | −0.436 |
| Urethane | ↑ | 0.751 | → | 0.025 | ↑ | 1.000 | → | 0.420 | → | −0.577 | ↑ | 0.578 |
| Ester | → | 0.566 | ↑ | 0.614 | → | 0.420 | ↑ | 1.000 | ↓ | −0.960 | → | −0.490 |
| Doublebond | ↓ | −0.774 | ↓ | −0.693 | → | −0.577 | ↓ | −0.960 | ↑ | 1.000 | → | 0.281 |
| BDO | → | 0.281 | → | −0.436 | → | 0.578 | → | −0.490 | → | 0.281 | ↑ | 1.000 |
| YoungMod | ↑ | 0.790 | ↑ | 0.770 | → | 0.270 | → | 0.207 | → | −0.426 | → | 0.182 |
| TensileStr | ↑ | 0.829 | ↑ | 0.819 | → | 0.407 | ↑ | 0.619 | ↓ | −0.756 | → | −0.087 |
| EaB | ↓ | −0.736 | ↓ | −0.781 | → | −0.204 | → | −0.224 | → | 0.421 | → | −0.100 |
| TearStr | ↑ | 0.903 | ↑ | 0.727 | → | 0.484 | → | 0.328 | → | −0.558 | → | 0.267 |
| ShoreA | ↑ | 0.787 | → | 0.295 | ↑ | 0.714 | → | 0.202 | → | −0.421 | → | 0.554 |

| | YoungMod | | TensileStr | | EaB | | TearStr | | ShoreA | |
|---|---|---|---|---|---|---|---|---|---|---|
| HardSeg | ↑ | 0.790 | ↑ | 0.829 | ↓ | −0.736 | ↑ | 0.903 | ↑ | 0.787 |
| Urea | ↑ | 0.770 | ↑ | 0.818 | ↓ | −0.781 | ↑ | 0.727 | → | 0.295 |
| Urethane | → | 0.270 | → | 0.407 | → | −0.204 | → | 0.484 | ↑ | 0.714 |
| Ester | → | 0.207 | ↑ | 0.619 | → | −0.224 | → | 0.328 | → | 0.202 |
| Doublebond | → | −0.426 | ↓ | −0.756 | → | 0.421 | → | −0.558 | → | −0.421 |
| BDO | → | 0.182 | → | −0.087 | → | −0.100 | → | 0.267 | → | 0.554 |
| YoungMod | ↑ | 1.000 | ↑ | 0.836 | ↓ | −0.943 | ↑ | 0.943 | ↑ | 0.711 |
| TensileStr | ↑ | 0.836 | ↑ | 1.000 | ↓ | −0.760 | ↑ | 0.833 | ↑ | 0.700 |
| EaB | ↓ | −0.943 | ↓ | −0.760 | ↑ | 1.000 | ↓ | −0.866 | ↓ | −0.614 |
| TearStr | ↑ | 0.943 | ↑ | 0.833 | ↓ | −0.866 | ↑ | 1.000 | ↑ | 0.800 |
| ShoreA | ↑ | 0.711 | ↑ | 0.700 | ↓ | −0.614 | ↑ | 0.800 | ↑ | 1.000 |

TABLE 5b

| | HardSeg | | Urea | | Urethane | | Ester | | Doublebond | | BDO | |
|---|---|---|---|---|---|---|---|---|---|---|---|---|
| HardSeg | ↑ | 1.000 | → | 0.647 | ↑ | 0.754 | → | 0.565 | ↓ | −0.774 | → | 0.281 |
| Urea | ↑ | 0.647 | ↑ | 1.000 | → | 0.024 | ↑ | 0.612 | ↓ | −0.689 | → | −0.437 |
| Urethane | ↑ | 0.754 | → | 0.024 | ↑ | 1.000 | → | 0.420 | → | −0.575 | → | 0.578 |
| Ester | → | 0.566 | ↑ | 0.612 | → | 0.420 | ↑ | 1.000 | ↓ | −0.960 | → | −0.490 |
| Doublebond | ↓ | −0.774 | ↓ | −0.689 | → | −0.578 | ↓ | −0.960 | ↑ | 1.000 | → | 0.281 |
| BDO | → | 0.281 | → | −0.437 | → | 0.578 | → | −0.490 | → | 0.281 | ↑ | 1.000 |
| YoungMod | ↑ | 0.650 | ↑ | 0.889 | → | 0.038 | → | 0.325 | → | −0.470 | → | −0.153 |
| TensileStr | ↑ | 0.711 | ↑ | 0.963 | → | 0.129 | ↑ | 0.800 | ↓ | −0.702 | → | −0.326 |
| EaB | ↓ | −0.701 | ↓ | −0.684 | → | −0.214 | → | −0.125 | → | 0.336 | → | −0.191 |
| TearStr | ↑ | 0.608 | ↑ | 0.820 | → | 0.300 | → | 0.363 | → | −0.553 | → | 0.059 |
| ShoreA | ↑ | 0.605 | → | 0.270 | ↑ | 0.751 | → | 0.183 | → | −0.413 | ↑ | 0.506 |

| | YoungMod | | TensileStr | | EaB | | TearStr | | ShoreA | |
|---|---|---|---|---|---|---|---|---|---|---|
| HardSeg | ↑ | 0.550 | ↑ | 0.711 | ↓ | −0.701 | ↑ | 0.808 | ↑ | 0.805 |
| Urea | ↑ | 0.589 | ↑ | 0.983 | ↓ | −0.594 | ↑ | 0.820 | → | 0.270 |
| Urethane | → | 0.038 | → | 0.129 | → | −0.214 | → | 0.300 | ↑ | 0.751 |
| Ester | → | 0.325 | → | 0.500 | → | −0.128 | → | 0.363 | → | 0.183 |
| Doublebond | → | −0.470 | ↓ | −0.702 | → | 0.336 | → | −0.553 | → | −0.413 |
| BDO | → | −0.153 | → | −0.326 | → | −0.191 | → | 0.059 | ↑ | 0.606 |
| YoungMod | ↑ | 1.000 | ↑ | 0. [symbol] | ↓ | −0.739 | ↑ | 0.950 | → | 0.408 |
| TensileStr | ↑ | 0. [symbol] | ↑ | 1.000 | ↓ | −0.595 | ↑ | 0.803 | → | 0.397 |
| EaB | ↓ | −0.739 | ↓ | −0. [symbol] | ↑ | 1.000 | ↓ | −0.746 | ↓ | −0.620 |
| TearStr | ↑ | 0.950 | ↑ | 0.503 | ↓ | −0.746 | ↑ | 1.000 | → | 0.570 |
| ShoreA | → | 0.408 | → | 0.397 | ↓ | −0.520 | → | 0.570 | ↑ | 1.000 |

[symbol]

indicates data missing or illegible when filed

TABLE 5b-2

| | M1 | | M2 | | M3 | | M4 | | M5 | | M6 | |
|---|---|---|---|---|---|---|---|---|---|---|---|---|
| M1 | ↑ | 1.000 | ↑ | 0.647 | ↑ | 0.754 | → | 0.566 | ↓ | −0.774 | → | 0.281 |
| M2 | ↑ | 0.647 | ↑ | 1.000 | → | 0.024 | ↑ | 0.612 | ↓ | −0.669 | → | −0.437 |
| M3 | ↑ | 0.754 | → | 0.024 | ↑ | 1.000 | → | 0.420 | → | −0.578 | → | 0.578 |
| M4 | → | 0.566 | ↑ | 0.612 | → | 0.420 | ↑ | 1.000 | ↓ | −0.980 | → | −0.490 |
| M5 | ↓ | −0.774 | ↓ | −0.689 | → | −0.578 | ↓ | −0.960 | ↑ | 1.000 | → | 0.281 |
| M6 | → | 0.281 | → | −0.437 | → | 0.578 | → | −0.490 | → | 0.281 | ↑ | 1.000 |
| P1 | ↑ | 0.650 | ↑ | 0.669 | → | 0.038 | → | 0.325 | → | −0.470 | → | −0.153 |
| P2 | ↑ | 0.711 | ↑ | 0.963 | → | 0.129 | ↑ | 0.800 | ↓ | −0.702 | → | −0.326 |
| P3 | ↓ | −0.701 | ↓ | −0.694 | → | −0.214 | → | −0.128 | → | 0.336 | → | −0.191 |
| P4 | ↑ | 0.806 | ↑ | 0.820 | → | 0.300 | → | 0.363 | → | −0.553 | → | 0.059 |
| P5 | ↑ | 0.605 | → | 0.270 | ↑ | 0.751 | → | 0.183 | → | −0.413 | ↑ | 0.606 |

| | P1 | | P2 | | P3 | | P4 | | P5 | |
|---|---|---|---|---|---|---|---|---|---|---|
| M1 | ↑ | 0.650 | ↑ | 0.711 | ↓ | −0.701 | ↑ | 0.808 | ↑ | 0.805 |
| M2 | ↑ | 0.889 | ↑ | 0.963 | ↓ | −0.694 | ↑ | 0.820 | → | 0.270 |
| M3 | → | 0.038 | → | 0.129 | → | −0.214 | → | 0.300 | ↑ | 0.751 |
| M4 | → | 0.325 | → | 0.600 | → | −0.128 | → | 0.363 | → | 0.183 |
| M5 | → | −0.470 | ↓ | −0.702 | → | 0.336 | → | −0.553 | → | −0.413 |
| M6 | → | −0.153 | → | −0.326 | → | −0.191 | → | 0.059 | ↑ | 0.806 |
| P1 | ↑ | 1.000 | ↑ | 0.859 | ↓ | −0.739 | ↑ | 0.950 | → | 0.406 |
| P2 | ↑ | 0.859 | ↑ | 1.000 | ↓ | −0.695 | ↑ | 0.803 | → | 0.397 |
| P3 | ↓ | −0.739 | ↓ | −0.695 | ↑ | 1.000 | ↓ | −0.746 | ↓ | −0.620 |
| P4 | ↑ | 0.950 | ↑ | 0.803 | ↓ | −0.746 | ↑ | 1.000 | → | 0.570 |
| P5 | → | 0.408 | → | 0.397 | ↓ | −0.620 | → | 0.570 | ↑ | 1.000 |

TABLE 3

| | YoungMod | | TensileStr | | EaB | | TearStr | | ShoreA | |
|---|---|---|---|---|---|---|---|---|---|---|
| HardSeg | ↑ | 0.790 | ↑ | 0.829 | ↓ | −0.736 | ↑ | 0.903 | ↑ | 0.805 |
| Urea | ↑ | 0.897 | ↑ | 0.968 | ↓ | −0.781 | ↑ | 0.853 | → | 0.295 |
| Urethane | → | 0.270 | → | 0.407 | → | −0.214 | → | 0.484 | ↑ | 0.751 |
| Ester | → | 0.325 | ↑ | 0.619 | → | 0.038 | → | 0.363 | → | 0.202 |
| Doublebond | → | −0.470 | ↓ | −0.756 | → | 0.421 | → | −0.558 | → | −0.444 |
| BDO | → | 0.182 | → | −0.326 | → | −0.292 | → | 0.267 | ↑ | 0.731 |

The following conclusions thus arise from table 3:
Modulus of elasticity (“YoungMod”) as behavior property is determined by the hard segment content (“HardSeg”) and the urea content (“Urea”)—each descriptors. Since both have a positive correlation, a higher hard segment and a higher urea content here means a higher modulus of elasticity.
The same applies to the behavior property of breaking stress (“TensileStr”), which additionally correlates positively with the ester content descriptor, while the double bond content descriptor (“Doublebond”) correlates negatively.
The behavior property of elongation at break (“EaB”) shows anti-proportional behavior, like the modulus of elasticity.
The behavior property of tear strength (“TearStr”) is correlated with the modulus of elasticity.
The behavior property of Shore A hardness (“Shore A”) correlates positively with the hard segment content (“HardSeg”), the urethane content (“Urethane”) and the descriptor of butanediol content (“BDO”), and is thus the sole behavior property considered that depends on the “BDO” descriptor.

The following dependences that affect optimization of the behavior properties are thus apparent:
Modulus of elasticity (“YoungMod”), elongation at break (“EaB”) and tear strength (“TensileStr”) affect one another.
Tear strength (“TensileStr”) can be increased by a high ester content and low double bond content (or else conversely lowered).
Shore A hardness (“Shore A”) is increased by high urethane content and high butanediol content.

The dependences of the behavior properties with respect to the descriptors are comprehended by evaluation of the correlation matrix and can then be adapted to target test feature values based on behavior properties according to a target profile of requirements of a target mixed product, for example in an application in paints, adhesives, sealing compounds, casting resins and foams. Suitable combinations of behavior properties can be inferred directly or even extrapolated from the datasets.

In the manner described, the method proposed enables systematic performance of product developments, and permits the developer of paints, adhesives, sealing compounds, casting resins and foams to find a targeted course of action and clear instructions for action for development of these products. In addition, it has been shown that behavior properties are dependent on particular (individual or multiple) descriptors, which permits buildup of knowledge for future tasks.

If tables 5a and 5a-2 and tables 5b and 5b-2 are considered in a comparative manner, the descriptors are cited there as M1-M6 and the behavior properties as P1-P5 in tables 5a-2 and 5b2. The content of the correlation matrices in tables 5b and 5b-2 is identical, and so it has been shown that, given additional coding of the descriptors and the behavior properties and the use of normalizing mappings (the student distribution here, for example), the assignment to particular parameters is not known, nor is it known what kind of parameter is involved. The numerical values that have been altered by the bijective mapping also means that it is not possible for the person skilled in the art to infer the correlation relationships to the descriptors or the behavior properties. But the content of the correlation matrix as instructions for action for the respective side (being aware either solely of descriptors or, on the other hand, solely of the behavior properties) is conserved.

It is to be shown hereinafter that it is possible by this course of action to calculate rationally derivable proposals for new product distributions of the product compositions of the examples given in table 1. For this purpose, reference is made firstly to table 4 in which the weight ratios of the product distributions from table 1 are given.

TABLE 4

| | IPDI | PCT | HTNR | BDO |
|---|---|---|---|---|
| Polyurethane 3 | 34.16 | 65.84 | 0.00 | 0.00 |
| Polyurethane 4 | 38.37 | 61.63 | 0.00 | 0.00 |
| Polyurethane 5 | 45.36 | 54.64 | 0.00 | 0.00 |
| Polvuresaate 6 | 48.29 | 51.71 | 0.00 | 0.00 |
| Polyurethane 7 | 19.78 | 19.06 | 61.15 | 0.00 |
| Polyurethane 8 | 25.40 | 17.14 | 54.97 | 2.49 |
| Polyurethane 9 | 31.34 | 15.10 | 48.43 | 5.13 |
| Polyurethane 10 | 47.01 | 45.30 | 0.00 | 7.69 |
| Polvurethane 11 | 36.16 | 24.39 | 33.53 | 5.92 |
| Polyurethane 12 | 27.65 | 7.99 | 59.83 | 4.52 |
| Polvurethane 13 | 23.50 | 0.00 | 72.65 | 3.85 |

For the calculation of table 4 from table 1, the equivalent weights of the four components IPDI: 110 g/mol, PCL: 265 g/mol, HTNR 850 g/mol and BDO 45 g/mol were used. These were multiplied by the equivalents figures given in table 1 and then converted to percent.

As already inferred from table 3, the important mechanical properties of modulus of elasticity and elongation at break are typical behavior properties to be optimized. The urea content descriptor and the hard segment content descriptor determine both behavior properties—except that the behavior property of modulus of elasticity does so in an inverse manner to the behavior property of elongation at break. In general, such a development task presents a problem to the developer since it is supposed that all he can do is seek a compromise.

In order then to achieve an improvement in both behavior properties, a graph representation is particularly suitable in a multivariate analysis. It can be important here to give greater consideration to the identification of the descriptors (here the urea content “Urea”) as important influencing factor on these two behavior properties. For instance, FIG. 1 shows modulus of elasticity (“YoungMod”) plotted against urea content (“Urea”), while FIG. 2 shows elongation at break (“EaB”) plotted against urea content (“Urea”). Both graphs show a defined target region marked in gray for which a new product distribution of the product composition of IPDI/PCL/HTNR/BDO is being sought and which thus constitutes a target profile of requirements for the mixed target product being sought. By comparison of the product compositions/product distributions within the gray target region, it becomes clear that no product from the selection of PU3 to PU13 fulfills the target profile of requirements described by the two target behavior properties.

FIG. 1 shows the strong positive correlation of urea content (“Urea”) with modulus of elasticity (“YoungMod”), for example from the example series PU3 to PU6 (PU=polyurethane). This was achieved in the example series by an increasing excess of equivalents of IPDI—shown by the vectors of PU3 to PU6—but with loss of elongation at break (“EaB”), as clearly shown by FIG. 2. The same applies to the example series PU7-PU9, where an increasingly higher use of BDO that likewise leads to a higher urea content through the low equivalents ratio likewise results in a higher modulus of elasticity—but at a lower level—which then also leads to a loss in elongation at break (“EaB”).

Using the same raw materials, PU10 is a possible starting point since this is already within the range of modulus of elasticity, but an increase in elongation at break is still required. The variation in the urea content from PU3 to PU4 leads to a small gain in modulus of elasticity and a high loss in elongation at break. Therefore, the reversed course of action should result in a small loss in modulus of elasticity and a large gain in elongation at break. In this way, a target descriptor profile is obtained. Proceeding from PU10, this should lead into the target region.

It is in turn possible to conclude the product composition from this descriptor profile. The change in the product distribution from PU4 to PU3 is the reduction in the equivalents ratio of isocyanate to alcohols. It follows as an instruction for action that, proceeding from the product composition PU10, the equivalents ratio should be reduced further by estimation from the graph with reference to the identification of the target feature value of the urea content descriptor “Urea”, or, in other words, the correlation matrix is utilized to alter the behavior properties in order to arrive via the descriptors at a target product composition of the mixed target product.

This is shown in the form of a graph in FIGS. 3 and 4, wherein the projection of the vectors in the same direction of the variation in behavior properties from PU3 to PU4 is used, but inverted in direction and roughly halved in length, in order to arrive within the target region shaded in gray. This already results in the new target composition based on PU10 which is shown in table A2. Polyurethanes 3, 4 and 10 are cited again as reference; the new mixed target product polyurethane 10-new shows the target behavior property with a graphically estimated urea content of 0.33. The equivalents ratio is calculated in such a way that the urea content is attained, such that the projection of the vectors in FIG. 6 still leads into the gray-shaded target region. The equivalents ratio of IPDI is thus chosen at 1.189 (which results in a urea content of 0.33) and, as shown above and elucidated for table 4, the new target distribution is calculated (see table 4-new).

TABLE A2
Derivation of the new equivalents ratios for the new target
composition Polyurethane 10-new (see also FIGS. 6 and 7)

| | IPDI | PCL | HTNR | BDO |
|---|---|---|---|---|
| Polyurethane 3 | 1.25 | 1.00 | 0.00 | 0.00 |
| Polyurethane 4 | 1.50 | 1.00 | 0.00 | 0.00 |
| Polyurethane 10 | 1.25 | 0.50 | 0.00 | 0.50 |
| Polyurethane 10-new | 1.19 | 0.50 | 0.00 | 0.50 |

TABLE 4-new
Result of the new product distribution of polyurethane 10-new

| | IPDI | PCL | HTNR | BDO |
|---|---|---|---|---|
| Polyurethane 10-new | 45.76 | 46.36 | 0.00 | 7.87 |

It has thus been shown that a graph analysis of the variations in behavior properties in relation to descriptors that have been identified beforehand as being of statistical relevance is a suitable course of action for predicting new product compositions and their product distributions. The term “projection” derives from the vectors that result from the graph assessment in the dimension space of behavior properties with respect to descriptors.

FIG. 5 shows a first 1, a second 3 and a third 5 computer system for execution of the proposed method of ascertaining a product composition for a mixed chemical product. The first computer system 1 is in a dedicated intranet 2, the second computer system 3 is likewise in a dedicated intranet 4, and the third computer system 5 is also in a dedicated intranet 6, with each intranet 2, 4, 6 being disjoint from the respective others. The three respectively disjoint intranets 2, 4, 6 may also each be referred to as first intranet 2, as second intranet 4 and as third intranet 6.

All three computer systems 1, 3, 5 are connected by the general internet 7, and there exist technical protective measures that enable exchange of information (especially: mapped feature values and mapped test feature values) between supplier and customer solely via their specific access

1.-15. (canceled)

16. A method of ascertaining a product composition for a mixed chemical product, wherein a multitude of feature values, each of which numerically describes a descriptor of the particular mixed product, is provided for each of a multitude of first product compositions for a particular mixed chemical product, wherein each first product composition is characterized by a numerical product distribution for description of proportions of components of the first product composition, wherein the series of feature values for each mixed product is mapped onto a series of mapped feature values by a first bijective mapping, wherein a series of test feature values, each of which numerically describes a behavior property of the particular mixed product, is provided for a multitude of second product compositions for a particular mixed chemical product, wherein the series of test feature values for each mixed product is mapped onto a series of mapped test feature values by a second bijective mapping, wherein at least the first or second mapping includes a variation, wherein each series of mapped test feature values of a second product composition is assigned to a series of varied feature values of a first product composition, wherein a multivariate analysis of the assigned series determines a correlation matrix, wherein a target profile of requirements for description of at least one behavior property of a target mixed product is defined and, on the basis of the target profile of requirements and the correlation matrix, a target descriptor profile for description of descriptors of a target product composition is determined.

17. The method as claimed in claim 16, wherein the determining of the target descriptor profile comprises, based on a comparison of the target descriptor profile with the feature values of the first product compositions of the multitude, determining a first product composition of the multitude as starting product composition and varying the product distribution of the starting product composition on the basis of the feature values of the remaining first product compositions of the multitude to obtain the target product composition.

18. The method as claimed in claim 16, wherein the series of varied test feature values of a first product composition is assigned to that series of varied feature values of a second product composition in which the second product composition is essentially identical to the first product composition.

19. The method as claimed in claim 16, wherein the series of feature values for each first product composition is provided on a first computer system on which the first bijective mapping is executed, in that the series of test feature values for each second product composition is provided on a second computer system on which the second bijective mapping is executed, and in that the first computer system and the second computer system are encompassed by a respectively disjoint intranet, preferably in that the determination of the target descriptor profile is performed at least partly on the first computer system.

20. The method as claimed in claim 19, wherein the multivariate analysis is executed on a third computer system encompassed by an intranet that is disjoint from the respective intranet of the first computer system and the second computer system.

21. The method as claimed in claim 19, wherein the product distribution and the series of feature values for each first product composition are stored by data encapsulation in the first computer system with respect to the second computer system, and in that the series of test feature values for each second product composition is stored with data encapsulation in the second computer system with respect to the first computer system.

22. The method as claimed in claim 19, wherein the series of varied feature values for each first product composition is transmitted from the first computer system to a target computer system in a disjoint intranet, preferably to the second computer system or the third computer system.

23. The method as claimed in claim 16, wherein, in a calculation model provided preferably in the first computer system, input of feature values for description of a particular descriptor results in output of a product distribution of a product composition for a mixed product for approximation of the feature values, and in that, preferably in the first computer system, the target descriptor profile is input into the calculation model for output of the target product composition.

24. The method as claimed in claim 23, wherein the calculation model is ascertained at least partly by multivariate analysis, preferably executed in the first computer system, of the series of feature values of each first product composition with respect to the product distribution of this product composition.

25. The method as claimed in claim 16, wherein, for each first product composition of the multitude, the series of feature values is ascertained at least partly, by a calculation based on the corresponding product distribution, preferably in that the calculation is based on a physical calculation model based on the product distribution.

26. The method as claimed in claim 16, wherein the first bijective mapping comprises a transformation of coordinates from the series of feature values to the series of varied feature values, and/or in that the second bijective mapping comprises a transformation of coordinates from the series of test feature values to the series of varied test feature values.

27. The method as claimed in claim 16, wherein the first bijective mapping comprises a one-dimensional bijective sub-mapping for each individual descriptor.

28. The method as claimed in claim 16, wherein the first bijective mapping or the second bijective mapping is a constant and strictly monotonous function with a continuously varying derivative.

29. A method of producing a mixed chemical product from a product composition, wherein the product composition has been ascertained by the method as claimed in claim 16.

30. A mixed chemical product, wherein the mixed chemical product has been produced by the method as claimed in claim 29.
